(19) United States
(12) Patent Application Publication
(10) Pub. No.: US 2002/0090621 A1
(43) Pub. Date: Jul. 11, 2002

(54) Combinatorial probes and uses therefor

(75) Inventors: Mark John Gibbs, Curtin (AU); Adrian John Gibbs, Yarralumla (AU); Roger William Brown, O'Connor (AU)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: The Australian National University, Acton (AU)

(21) Appl. No.: 09/916,808

(22) Filed: Jul. 27, 2001

Related U.S. Application Data
(60) Provisional application No. 60/226,212, filed on Aug. 18, 2000.

(30) Foreign Application Priority Data
Jul. 27, 2000 (AU) PQ 9026
Aug. 17, 2000 (AU) PQ 9483

(51) Int. Cl. C12Q 1/68; G06F 19/00; G01N 33/48; G01N 33/50; C07H 21/04

(52) U.S. Cl. 435/6; 702/20; 536/24.3

(57) ABSTRACT

A set of oligonucleotide probes and method are disclosed for detecting a plurality of different target polynucleotides. The set includes a collection of different promiscuous probes each of which is capable of hybridizing to a target sequence shared between at least two of the target polynucleotides. At least one target polynucleotide comprises at least one target sequence that is shared with one or more other target polynucleotides. A predefined combination of promiscuous probes is capable of hybridizing to target sequences of said at least one target polynucleotide, wherein said predefined combination of probes provides specificity of detection of that target polynucleotide. Also disclosed are processes of identifying a set of target sequences for designing the set of oligonucleotide probes of the invention.

THIS INVENTION relates generally to novel means and methods for nucleic acid analysis and detection. More particularly, the present invention relates to a set of oligonucleotide probes, wherein two or more probes, in combination, can specifically detect a target polynucleotide and wherein different combinations of probes provide specificity for detecting and distinguishing different target polynucleotides. The invention also relates to methods for designing such combinations of oligonucleotide probes by way of gene sequence analyses that are preferably carried out using a digital computer, and to methods for interpreting the results of tests using such probe combinations.

### BACKGROUND OF THE INVENTION

Modem societies require accurate identification of biological organisms or their parts for a whole range of crucial reasons, including the diagnosis, understanding and control of diseases, quarantine control and industrial processes, etc. Techniques based on nucleic acid hybridisation are unparalleled in their ability to identify and quantify the genetic material (DNA or RNA) of particular organisms or groups of genetically related organisms. The provision of multiplexed (parallelised) assays, such as DNA microfabricated arrays (micro-arrays), now allows an ‘order of magnitude’ increase in speed and specificity for this kind of gene-based analysis. For example, reference may be made to Southern (WO89/10977; U.S. Pat. No. 6,045,270), Chee et al. (U.S. Pat. No. 5,837,832) Cantor et al. (U.S. Pat. No. 6,007,987), and Fodor et al. (U.S. Pat. No. 5,871,928). Analogous multiplexed arrays are obtained using microbeads and their assay by flow cytometry (Cai H, et al., 2000, Genomics 66: 135-43 (ibid Erratum 69: 395)).

Until recently the nucleic acid probes used in nucleic acid hybridisations were mostly obtained empirically by isolating DNA or RNA fragments that were derived from the targeted organism(s) or gene(s). However, it is now possible to design and synthesise nucleic acid probes using data from the international sequence databases (e.g., the GenBank and EMBL databases). These databases of known gene sequences have been increasing tenfold in size every five years for many years and now contain a representative sample of most genes and most major groups of organisms.

Generally, DNA micro-arrays use spots of detector oligonucleotides or probes positioned in arrays on a solid support, typically a glass wafer. The probes are allowed to hybridise with sample nucleic acids, which contain the target nucleic acids and which have been fluorescently labelled. The probes and target nucleic acids of the sample are allowed to hybridise under conditions that only detect exact or almost exact complementarity between the probes and the target nucleic acids. If a target nucleic acid complements and hybridises to a particular probe in the array, the spot will fluoresce. Recording the fluorescence of the spots enables one to assess which target sequences are present in the nucleic acids mixture.

Sequence information, obtained from native RNA or DNA molecules, is used to determine the sequence of the synthesised oligonucleotide probes and this information is usually stored in computer databases and manipulated using software. Each probe is synthesised so that it contains nucleotides in an order (sequence) that matches a part of a known native nucleotide sequence or the complement of a part of that sequence. Oligonucleotide probes used in conventional arrays are typically 10-25 nucleotides long. For the purposes of the present invention, and as will be more fully discussed hereinafter, the nucleic acid molecules that are to be identified in an assay or test are designated “target polynucleotides”. The parts or segments of these polynucleotides that match the sequence of, and hybridise to, an oligonucleotide probe are designated “target sequences”. This term also includes within its scope sequences as represented in a computer datafile or some other readable form.

Currently oligonucleotide probes are most commonly used in micro-arrays to identify and quantify the mRNA transcripts from genes. These micro-arrays usually contain probes representing several different target sequences from each gene sequence and these probes are usually chosen to be target specific (i.e., they hybridise with just one target polynucleotide). Thus, these micro-arrays contain many more probes than the number of target polynucleotides they are designed to detect.

Compared to conventional nucleic acid analysis techniques including restriction fragment length polymorphism (RFLP) analysis and the polymerase chain reaction (PCR), DNA micro-arrays provide a facile and rapid means of detecting and measuring the expression of different genes. They have also been used to detect variants of well-characterised nucleic acid molecules (i.e., to detect genetic polymorphisms and genotypes). However, despite their promise as tools for diagnosing infectious diseases as well as genetic disorders, the development of micro-arrays for routine diagnosis appears to be slow. This is probably due to the relatively high cost of designing, developing and producing micro-arrays that could detect a large number of target polynucleotides. New methods and reagents are, therefore, required to realise this promise, and the present invention helps to meet that need. The present invention provides improved nucleic acid analysis techniques as described more fully hereinafter.

### BRIEF SUMMARY OF THE INVENTION

Accordingly, in one aspect of the invention, there is provided a set of oligonucleotide probes for detecting a plurality of different target polynucleotides, wherein a respective target polynucleotide corresponds to a single polynucleotide or a group of related polynucleotides, said set including a collection of different promiscuous probes, wherein a respective promiscuous probe is capable of hybridising to a target sequence shared between at least two of said target polynucleotides, wherein at least one target polynucleotide comprises at least two target sequences shared between other target polynucleotides, and wherein a predefined combination of promiscuous probes is capable of hybridising to said at least two target sequences, said predefined combination providing specificity of detection of said at least one target polynucleotide.

Preferably, the set of oligonucleotide probes comprises a plurality of different predefined combinations of probes, each providing specificity of detection of a different target polynucleotide.

In one embodiment, the set of oligonucleotide probes further comprises at least one non-promiscuous probe that is capable of hybridising to a unique target sequence of a single target polynucleotide.

In another embodiment, the set of oligonucleotide probes comprises at least one probe that is capable of hybridising to a pivot sequence, which divides two or more polynucleotides into distinct groups.

In yet another embodiment, the set of oligonucleotide probes comprises at least one degenerate oligonucleotide probe that is capable of hybridising to a redundant target sequence.

In another aspect, the invention provides a method for detecting a plurality of different target polynucleotides using the set of oligonucleotide probes as broadly described above, said method comprising:

exposing said probes to a test sample suspected of containing one or more of said target polynucleotides under stringent hybridisation conditions;

detecting which probes have hybridised to polynucleotides in said test sample; and

processing the hybridisation data to determine which of said predefined combinations of probes has hybridised to said polynucleotides to thereby determine whether the test sample comprises any of said target polynucleotides.

Preferably, the method further comprises analysing whether any of said target polynucleotides in said test sample corresponds to a phenotype-determining target polynucleotide.

Suitably, the method further comprises diagnosing a phenotype of a patient from which said test sample was derived based on the phenotype-determining target polynucleotide(s) present in the test sample.

In a preferred embodiment, the step of processing is performed by a programmable digital computer.

In yet another aspect, the invention provides a method for detecting an unknown or uncharacterised member of a polynucleotide family using the set of probes as broadly described above, said method comprising:

exposing said probes to a test sample under stringent hybridisation conditions;

detecting which probes have hybridised to polynucleotides in said test sample; and

processing the hybridisation data to determine which combinations of probes have hybridised to polynucleotides in said test sample, and whether any of said combinations is different to at least one predefined combination of probes that hybridise to known target sequences, wherein the presence of a different combination of oligonucleotide probes is indicative of the presence of said unknown or uncharacterised member.

Preferably, the different combination of oligonucleotide probes corresponds to a hypothetical predefined combination of probes belonging to a predefined assemblage.

Suitably, the hypothetical predefined combination of probes comprises at least one degenerate oligonucleotide probe that is capable of hybridising to a redundant target sequence.

In a further aspect of the invention, there is provided a process of identifying a set of target sequences from a plurality of known target polynucleotides for designing a set of oligonucleotide probes as broadly described above, said process comprising:

searching a nucleic acid sequence database comprising the sequences of a plurality of target polynucleotides for identical target sequences that are shared between two or more of said target polynucleotides to thereby obtain a subset of shared target sequences; and

determining for each target polynucleotide a combination of target sequences from said subset which, when hybridised by complementary or substantially complementary oligonucleotide probes, facilitate specific detection of that target polynucleotide.

In a preferred embodiment, the process further includes the step of:

sorting the target sequences from said subset to obtain pivot sequences which divide two or more polynucleotides into distinct groups.

Suitably, said process further comprises:

determining a minimal or near minimal number of promiscuous oligonucleotide probes which, in different combinations, discriminate between the different target polynucleotides.

In an alternate embodiment, the process preferably comprises:

searching the database for sequences that are unique to respective target polynucleotides to thereby obtain a subset of unique target sequences; and

determining for each target polynucleotide a target sequence from said unique subset, or a combination of target sequences from said shared subset and/or said unique subset which, when hybridised by complementary or substantially complementary oligonucleotide probe(s), facilitate(s) specific detection of that target polynucleotide.

Suitably, said process further comprises:

determining a minimal or near minimal number of promiscuous probes which, in different combinations, together with one or more non-promiscuous probes, discriminate between the different target polynucleotides.

In another embodiment, the process suitably comprises:

searching the database for target sequences that are substantially identical or conserved between related target polynucleotides; and

deducing redundant sequences corresponding to potential sequence variants of said target sequences to thereby obtain a subset of redundant target sequences which correspond to potentially unknown or uncharacterised target polynucleotides; and

determining for each target polynucleotide a target sequence from said redundant subset, or a combination of target sequences from said shared subset and/or said redundant subset which, when hybridised by complementary or substantially complementary oligonucleotide probe(s), facilitate(s) specific detection of that target polynucleotide.

Suitably, the process comprises:

sorting target sequences from one or more of said subsets to obtain target sequences with substantially similar affinities for their complementary or substantially complementary oligonucleotide probes.

Preferably, the process comprises:

sorting the target sequences from said redundant subset, from said shared subset and optionally from said unique subset to obtain target sequences with substantially similar affinities for their complementary or substantially complementary promiscuous or non-promiscuous oligonucleotide probes.

Preferably, said process is performed by a digital computer.

In yet another aspect, the invention provides a computer program product for identifying a set of target sequences for designing a set of oligonucleotide probes, as broadly described above, comprising code that receives as input sequences of target polynucleotides from one or more nucleic acid sequence databases and/or information that identifies sequences corresponding to said target polynucleotides; code that identifies potential target sequences within the target polynucleotides; code that identifies the target sequences that are shared between different target polynucleotides; optional code that identifies the target sequences that are unique to specific target polynucleotides, code that assesses every possible combination or a number of combinations of the target sequences to identify those combinations of target sequences which, when hybridised by complementary oligonucleotide probes, facilitate discrimination between different target polynucleotides; and a computer readable medium that stores the codes.

Suitably, the computer program product further comprises code that creates a database which registers the presence or absence of possible target sequences found within respective target polynucleotides.

Preferably, the computer program product further comprises code that identifies substantially identical or conserved sequences between the target sequences and code that identifies redundant sequence variants of said substantially identical target sequences, wherein said redundant sequence variants are registered as target sequences.

In yet another aspect, the invention provides a computer program product for processing hybridisation data comprising code that identifies for each target polynucleotide a combination of features in an oligonucleotide array whose probes facilitate specific detection of that polynucleotide; code that receives as input hybridisation data from hybridisation reactions between sample polynucleotides and the oligonucleotide probes in the array; code that processes the hybridisation data to determine whether the sample polynucleotides comprise any of the target polynucleotides by searching for hybridisation patterns that match any of the predefined combinations or predefined assemblages of target sequences; and a computer readable medium that stores the codes.

Preferably, said computer program product comprises code that receives as input the sequence of an oligonucleotide probe in each feature of an oligonucleotide array and code that receives as input a database that contains information on the presence or absence of target sequences in target polynucleotides.

Preferably the computer program product further comprises code that deduces the probability that the detected pattern of hybridisation indicates the presence of a target polynucleotide.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 shows a hypothetical target sequence and the set of all possible sub-sequences including eight or more bases derived from the target sequence.

FIG. 2A shows a Venn diagram representing the relationships between the sub-sequence of three hypothetical target sequences (A, B and C). Some sub-sequences derived from each target sequence are unique and some are shared. Target A shares some sub-sequence with B and some with C and some with both B and C, and C and B share some that are not shared with A.

FIG. 2B shows a Venn diagram matching FIG. 2A and showing which sub-sequences (X and Y) could be used to reduce the size of the set required to detect and distinguish between targets A, B and C.

FIG. 3 shows the sequence of the shared ‘B-motif’ in potyvirus polymerase genes. Positions (sites) in the sequence where variations are found are boxed, and each box lists the different nucleotides known to occur at that site.

FIG. 4 is a diagrammatic representation of an array of oligonucleotides. Each square (feature) on the grid represents a different oligonucleotide spot on an array consisting of 256 different oligonucleotides. Every possible combination of the sequence variants shown in FIG. 3 is represented in one of the 256 spots on the array. The spots on the array could be ordered so that the oligonucleotides in the rows and columns identified with arrows carry the sequence variations as shown for positions 3, 6 and 9. Oligonucleotides with variations in position 12, 15 and 18 could be similarly identified.

FIG. 5 comprises FIGS. 5A-5C and is a diagrammatic representation showing the expected reactions on an array designed as shown in FIG. 4 when DNAs encoding the polymerase B-motifs of the potyviruses potato virus Y (PVY) and bean yellow mosaic (BYMV) are used. The nucleotides at variable positions 3 and 6 (see FIG. 3) are shown to the left of the array and those at variable positions 9, 12 and 15 are shown above the array. The reactions with cDNA generated from the RNA of three groups of potyviruses are shown: FIG. 5A: strains-N (GenBank code D00441), -NFR (X12456) and -PA (A08776); FIG. 5B: strains-Hung (M495491) and -NSW (X97895); and FIG. 5C: strain-CO (U09509) and also BYMV strain S (U47033), but not-MB (D83749).

FIG. 6 comprises FIGS. 6A-6D and is a diagrammatic representation depicting shared gene sequences in potyvirus genomes showing sequence variations present in those sequences, and the overlapping parts of two of those sequences that could be used combinatorially as probes in a micro-array to detect and identify potyviruses. FIG. 6A: A region of the polymerase encoding its ‘B-motif’, and two sub-sequences derived from it; FIG. 6B: A region of the polymerase encoding its ‘B-motif’ and three sub-sequences derived from it; FIG. 6C: A region of the virion protein gene encoding the ‘WCIEN-motif’, and two sub-sequences of it; FIG. 6D: A region of the cylindrical inclusion protein encoding the ‘NVED-motif’.

FIG. 7 is a diagrammatic representation depicting the pattern of permutations of variable sites in the probes designed from three conserved regions of potyvirus genomes (FIG. 6). Each square in each grid is equivalent to a spot on the array that would carry a different oligonucleotide. The nucleotides at variable positions in the sequences are shown above and to the left of the grids/arrays.

FIG. 8 is a diagrammatic representation depicting hybridisation patterns obtained using copies of a hypothetical micro-array to detect cDNAs encoding the genomes of six different strains of potato virus Y and one of bean yellow mosaic virus (BYMV-S). The probes were 11-13 nucleotides long and had the sequences shown in FIG. 7. The virus-derived cDNAs match those in the example shown in FIG. 5.

FIG. 9 is a diagrammatic representation of a system used to carry out the instructions encoded by the storage medium of FIGS. 11 and 12.

FIG. 10 depicts a flow diagram showing an embodiment of a method for designing combinatorial probes according to the present invention.

FIG. 11 is a diagrammatic representation showing a cross section of a magnetic storage medium.

FIG. 12 is a diagrammatic representation showing a cross section of an optically readable data storage medium.

### DESCRIPTION OF THE SEQUENCES: SUMMARY TABLE

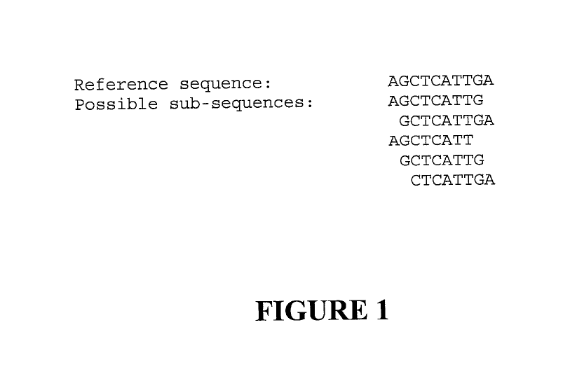

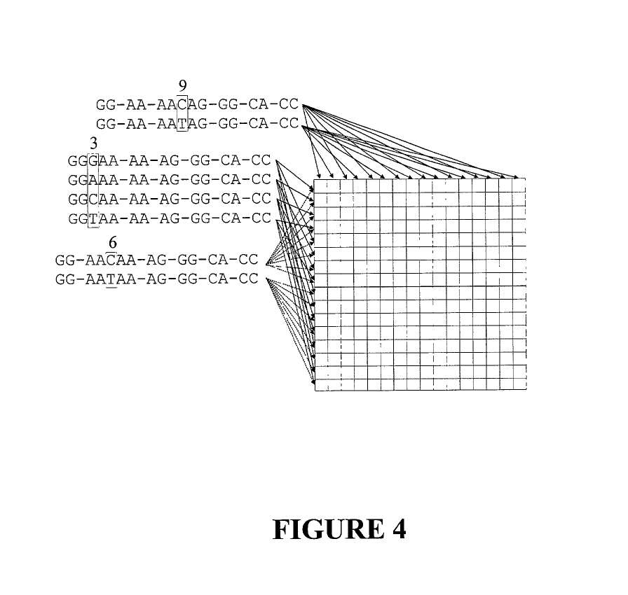

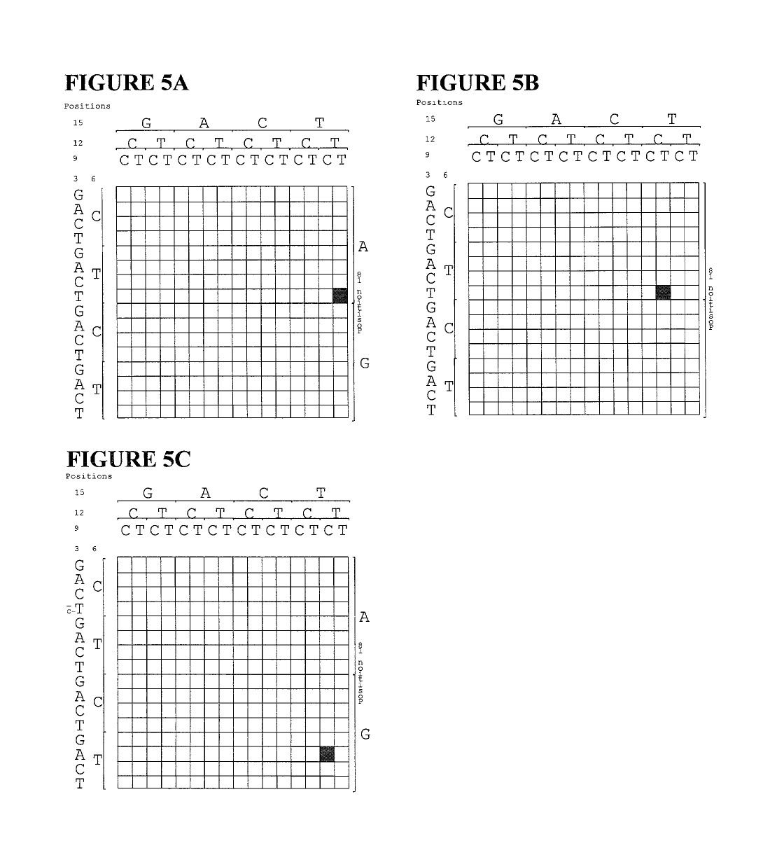

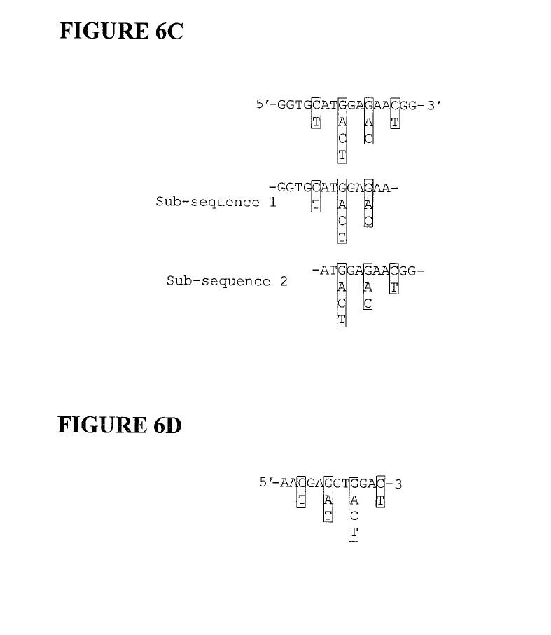

TABLE A

| SEQUENCE ID | | |
|---|---|---|
| NUMBER | SEQUENCE | LENGTH |
| SEQ ID NO: 1 | Reference sequence, FIG. 1 | 10 nts |
| SEQ ID NO: 2 | First putative sub-sequence, FIG. 1 | 9 nts |
| SEQ ID NO: 3 | Second putative sub-sequence, FIG. 1 | 9 nts |
| SEQ ID NO: 4 | Third putative sub-sequence, FIG. 1 | 8 nts |
| SEQ ID NO: 5 | Fourth putative sub-sequence, FIG. 1 | 8 nts |
| SEQ ID NO: 6 | Fifth putative sub-sequence, FIG. 1 | 8 nts |
| SEQ ID NO: 7 | Degenerate probe, FIG. 3 | 20 nts |
| SEQ ID NO: 8 | First probe, FIG. 4 | 15 nts |
| SEQ ID NO: 9 | Second probe, FIG. 4 | 15 nts |
| SEQ ID NO: 10 | Third probe, FIG. 4 | 15 nts |
| SEQ ID NO: 11 | Fourth probe, FIG. 4 | 15 nts |
| SEQ ID NO: 12 | Fifth probe, FIG. 4 | 15 nts |
| SEQ ID NO: 13 | Sixth probe, FIG. 4 | 15 nts |
| SEQ ID NO: 14 | Seventh probe, FIG. 4 | 15 nts |
| SEQ ID NO: 15 | Eighth probe, FIG. 4 | 15 nts |
| SEQ ID NO: 16 | Reference sequence, FIG. 6A | 20 nts |
| SEQ ID NO: 17 | First sub-sequence, FIG. 6A | 14 nts |
| SEQ ID NO: 18 | Second sub-sequence, FIG. 6A | 17 nts |
| SEQ ID NO: 19 | Reference sequence, FIG. 6B | 20 nts |
| SEQ ID NO: 20 | First sub-sequence, FIG. 6B | 11 nts |
| SEQ ID NO: 21 | Second sub-sequence, FIG. 6B | 11 nts |
| SEQ ID NO: 22 | Third sub-sequence, FIG. 6B | 11 nts |
| SEQ ID NO: 23 | Reference sequence, FIG. 6C | 16 nts |
| SEQ ID NO: 24 | First sub-sequence, FIG. 6C | 13 nts |
| SEQ ID NO: 25 | Second sub-sequence, FIG. 6C | 11 nts |
| SEQ ID NO: 26 | Reference sequence, FIG. 6D | 12 nts |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

The term “complementary” refers to the topological capability or matching together of interacting surfaces of an oligonucleotide probe and its target oligonucleotide, which may be part of a larger polynucleotide. Thus, the target and its probe can be described as complementary, and furthermore, the contact surface characteristics are complementary to each other. Complementary includes base complementarity such as A is complementary to T or U, and C is complementary to G in the genetic code. However, this invention also encompasses situations in which there is non-traditional base-pairing such as Hoogsteen base pairing which has been identified in certain transfer RNA molecules and postulated to exist in a triple helix. In the context of the definition of the term “complementary”, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that hybridise less efficiently.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The term “degenerate oligonucleotide probes” refers to a set of probes having substantially similar sequences, some of which match known, preferably conserved, target sequences and some of which are similar but not identical to the same known target sequences. These latter target sequences correspond to redundant target sequences as defined herein. Oligonucleotides probes that recognise redundant target sequences contain sequence variations that exist in at least two of the known target sequences but not together in one sequence, i.e., they match one of these sequences at one nucleotide position but at least one other known target sequence at another nucleotide position. Thus, these probe sets contain potential permutations of known sequence variants that have not yet been reported but are likely to occur in nature.

The term “feature” refers to an area of a substrate having a collection of substantially same-sequence, surface immobilised oligonucleotide probes. Generally, one feature is different from another feature if the probes of the different features have substantially different nucleotide sequences. In the context of light-directed oligonucleotide synthesis, for example, a feature is a spatially addressable synthesis site as for example disclosed in U.S. Pat. Nos. 5,384,261; 5,143,854; 5,150,270; 5,593,139; 5,634,734; and WO95/11995.

By “gene” is meant a genomic nucleic acid sequence at a particular genetic locus.

The term “gene family” or “family of polynucleotides” refers to a set of polynucleotides or genes or the polypeptides they encode, that have statistically significant sequence homology as, for example, determined by appropriate Monte Carlo shuffling tests (Hunter and Kearney, 1983, Biol Cybern 47(2): 141-146). Such sets are related through common ancestry as a result of gene inheritance by related but separate lineages or by gene duplication or by horizontal gene transfer or an equivalent recombinational process and subsequent evolution. Such sets include nucleic acid species from related pathogens, such as different genotypes or strains of a bacterial or virus species or different bacterial or viral species belonging to a single genus. Such sets also include genes that share a region that encodes a related domain. Many shared sequences encoding domains are known in the art including, for example, the ATPase domain, the cadherin-like domain, the EGF domain, the immunoglobulin domain, and the fibronectin type II domain. Reference may be made in this respect to R. F. Doolittle (1995, Annu. Rev. Biochem. 64: 287-314). Gene families frequently encode polypeptides sharing conserved regions, but may also include conserved regions that encode RNA that interact with other polynucleotides, and regions that interact with proteins, such as homeobox and tymobox regions. Conserved regions may extend to those in intronic sequences and genomic regions whose functions are currently unknown. By way of example, polypeptides share a highly conserved region if the polypeptides have a sequence identity of at least 60% over a comparison window of ten amino acids, or if they share a sequence identity of at least 80% over a comparison window of at least five amino acids.

By “high density polynucleotide arrays” and the like is meant those arrays that contain at least 400 different features per cm².

The phrase “high discrimination hybridisation conditions” refers to hybridisation conditions in which single base mismatch may be determined.

The phrase “hybridising specifically to” and the like refer to the binding, duplexing, or hybridising of a molecule only to a particular nucleotide sequence under stringent conditions when that sequence is present in a complex mixture (e.g., total cellular) DNA or RNA.

By “minimal number of probes” is meant the theoretical minimal number of probes described by the formulae X=log₂Y where X is the number of probes and Y is the number of target polynucleotides to be distinguished by those probes.

By “near-minimal number of probes” is meant a number of probes that is less than the number of target polynucleotides but greater than the minimal number of probes. Preferably a near-minimal number of probes would be less than 50% of the number of target polynucleotides, but more preferably less than 40%, less than 30%, less than 20%, less than 10%, or less than 5%.

By “obtained from” is meant that a sample such as, for example, a polynucleotide extract is isolated from, or derived from, a particular source of the host. For example, the extract can be obtained from a tissue or a biological fluid isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds, or related structural variants or synthetic analogues thereof, such as ‘locked nucleic acids’ (e.g., conformationally restricted nucleotide analogues with an extra 2′-O,4′-C-methylene bridge added to the ribose ring; Christensen U, et al., 2001, Biochem J 354: 481-4). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 8 to 30 nucleotides, more preferably from about 10 to 20 nucleotides and still more preferably from about 11 to 17 nucleotides, but the term can refer to molecules of any length, although the term “polynucleotide” or “nucleic acid” is typically used for large oligonucleotides. Oligonucleotides may be prepared using any suitable method, such as, for example, the phosphotriester method as described in an article by Narang et al. (1979, Methods Enzymol. 68 90) and U.S. Pat. No. 4,356,270. Alternatively, the phosphodiester method as described in Brown et al. (1979, Methods Enzymol. 68 109) may be used for such preparation. Automated embodiments of the above methods may also be used. For example, in one such automated embodiment, diethylphosphoramidites are used as starting materials and may be synthesised as described by Beaucage et al. (1981, Tetrahedron Letters 22 1859-1862). Reference also may be made to U.S. Pat. Nos. 4,458,066 and 4,500,707, which refer to methods for synthesising oligonucleotides on a modified solid support. It is also possible to use a primer, which has been isolated from a biological source (such as a denatured strand of a restriction endonuclease digest of plasmid or phage DNA). In a preferred embodiment, the oligonucleotide is synthesised according to the method disclosed in U.S. Pat. No. 5,424,186 (Fodor et al.). This method uses lithographic techniques to synthesise a plurality of different oligonucleotides at precisely known locations on a substrate surface.

The term “oligonucleotide array” refers to a substrate having oligonucleotide probes with different known sequences deposited at discrete known locations associated with its surface. For example, the substrate can be in the form of a two dimensional substrate as described in U.S. Pat. No. 5,424,186. Such substrate may be used to synthesise two-dimensional spatially addressed oligonucleotide (matrix) arrays. Alternatively, the substrate may be characterised in that it forms a tubular array in which a two dimensional planar sheet is rolled into a three-dimensional tubular configuration. The substrate may also be in the form of a microsphere or bead connected to the surface of an optic fibre as, for example, disclosed by Chee et al. in WO 00/39587. Oligonucleotide arrays have at least two different features and a density of at least 400 features per cm2. In certain embodiments, the arrays can have a density of about 500, at least one thousand, at least 10 thousand, at least 100 thousand, at least one million or at least 10 million features per cm2. For example, the substrate may be silicon or glass and can have the thickness of a glass microscope slide or a glass cover slip, or may be composed of other synthetic polymers. Substrates that are transparent to light are useful when the method of performing an assay on the substrate involves optical detection. The term also refers to a probe array and the substrate to which it is attached that form part of a wafer.

The term “patient” refers to patients of any animal origin, including humans, and includes any individual it is desired to examine or treat using the methods of the invention. However, it will be understood that “patient” does not imply that symptoms are present.

By “phenotype-determining target polynucleotide” is meant a target polynucleotide that is associated with a particular phenotype of an organism including, but not restricted to, a disease or condition.

The term “pivot sequence” is used herein to refer to a target sequence that occurs in two or more of the target polynucleotides but not in all of the target polynucleotides. Preferably a pivot sequence occurs in about 20% to about 80% of target polynucleotides, more preferably in about 30% to about 70%, more preferably in about 40% to about 60% and more preferably in about 45% to about 55% of the chosen target polynucleotides.

The term “predefined assemblage” refers to a collection of oligonucleotide probes that is made up of members which belong to two or more predefined sets of oligonucleotide probes, wherein oligonucleotides probes from these predefined sets are at least substantially complementary to, and would be expected to hybridise with, a family or group of related target polynucleotides. For example, the presence of a target polynucleotide may be indicated by hybridisation with oligonucleotide probes from several predefined sets, but it may not be known before hand to which oligonucleotide probes in each set the target polynucleotide will hybridise. A predefined assemblage preferably contains degenerate oligonucleotide probes as defined herein.

The term “predefined combination” refers to a combination of oligonucleotide probes that are at least substantially complementary to, or would be expected to hybridise with, target sequences of a single target polynucleotide. Target sequences which are recognised by a predefined combination of probes encompass known target sequences or a potential or hypothetical combination of at least one known target sequence and at least one redundant target sequence as defined herein. Such potential combination of target sequences can be recognised by oligonucleotide probes belonging to a predefined assemblage as described hereinafter.

“Probe” refers to an oligonucleotide molecule that binds to a specific target sequence or other moiety of another nucleic acid molecule. Unless otherwise indicated, the term “probe” in the context of the present invention typically refers to an oligonucleotide probe that binds to another oligonucleotide or polynucleotide, often called the “target polynucleotide”, through complementary base pairing. Probes can bind target polynucleotides lacking complete sequence complementarity with the probe, depending on the stringency of the hybridisation conditions. Oligonucleotide probes may be selected to be “substantially complementary” to a target sequence as defined herein. The exact length of the oligonucleotide probe will depend on many factors including temperature and source of probe and use of the method. For example, depending upon the complexity of the target sequence, the oligonucleotide probe may typically contain 8 to 30 nucleotides, more preferably from about 10 to 20 nucleotides and still more preferably from about 11 to 17 nucleotides capable of hybridisation to a target sequence although it may contain more or fewer such nucleotides.

The term “redundant target sequence” refers a hypothetical or potential target sequence that has been deduced from substantially identical or conserved target polynucleotides. The deduced sequences may therefore correspond to potential permutations of known sequence variants, which have not yet been reported but are likely to occur in nature. For example, redundant target sequences may be deduced from reference sequences of a gene family. This term also includes within its scope sequences as represented in a computer datafile or some other readable form that could be used to guide the synthesis of redundant oligonucleotide probes.

By “reference sequence” is meant a part or segment of a target polynucleotide that could be used to guide the selection of a target sequence.

Terms used to describe sequence relationships between two or more polynucleotides or polypeptides include “comparison window”, “sequence identity”, “percentage of sequence identity” and “substantial identity”. Because two polynucleotides may each comprise (1) a sequence (i.e., only a portion of the complete polynucleotide sequence) that is similar between the two polynucleotides, and (2) a sequence that is divergent between the two polynucleotides. Sequence comparisons between two (or more) polynucleotides are typically performed by comparing sequences of the two polynucleotides over a “comparison window” to identify and compare local regions of sequence similarity. A “comparison window” refers to a conceptual segment of at least 20 contiguous positions, usually about 20 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, WI, USA; CLUSTAL described by Jeanmougin, F., et al., 1998, Trends Biochem. Sci. 23: 403-5) or by inspection, or using dot diagrams, and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25: 3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, California, USA) using standard defaults as used in the reference manual accompanying the software.

“Stringency” as used herein refers to the temperature and ionic strength conditions, and presence or absence of certain organic solvents, during hybridisation. The higher the stringency, the higher will be the observed degree of complementarity between immobilized polynucleotides and the labelled target polynucleotide.

“Stringent conditions” as used herein refers to temperature and ionic conditions under which only polynucleotides having a high proportion of complementary bases, preferably having exact complementarity, will hybridise. The stringency required is nucleotide sequence dependent and depends upon the various components present during hybridisation, and is greatly changed when nucleotide analogues are used. Generally, stringent conditions are selected to be about 10 to 20° C. less than the thermal melting point (Tₘ) for the specific sequence at a defined ionic strength and pH. The Tₘ is the temperature (under defined ionic strength and pH) at which 50% of a target sequence hybridises to a complementary probe. It will be understood that an oligonucleotide probe will hybridise to a target sequence under at least low stringency conditions, preferably under at least medium stringency conditions and more preferably under high stringency conditions. Reference herein to low stringency conditions include and encompass from at least about 1% v/v to at least about 15% v/v formamide and from at least about 1 M to at least about 2 M salt for hybridisation at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at room temperature. Medium stringency conditions include and encompass from at least about 16% v/v to at least about 30% v/v formamide and from at least about 0.5 M to at least about 0.9 M salt for hybridisation at 42° C., and at least about 0.5 M to at least about 0.9 M salt for washing at 42° C. Medium stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at 42° C. High stringency conditions include and encompass from at least about 31% v/v to at least about 50% v/v formamide and from at least about 0.01 M to at least about 0.15 M salt for hybridisation at 42° C., and at least about 0.01 M to at least about 0.15 M salt for washing at 42° C. High stringency conditions also may include 1% BSA, 1 mM EDTA, 0.5 M NaHPO₄ (pH 7.2), 7% SDS for hybridisation at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. Other stringent conditions are well known in the art. A skilled addressee will recognise that various factors can be manipulated to optimise the specificity of the hybridisation. Optimisation of the stringency of the final washes can serve to ensure a high degree of hybridisation. For detailed examples, see Ausubel et al., supra at pages 2.10.1 to 2.10.16 and Sambrook et al. (1989, supra) at sections 1.101 to 1.104.

By “substantially complementary” it is meant that an oligonucleotide probe is sufficiently complementary to hybridise with a target sequence. Accordingly, the nucleotide sequence of the oligonucleotide probe need not reflect the exact complementary sequence of the target sequence. In a preferred embodiment, the oligonucleotide probe contains no mismatches and with the target sequence.

The phrase “substantially similar affinities” refers herein to target sequences having similar strengths of detectable hybridisation to their complementary or substantially complementary oligonucleotide probes under a chosen set of stringent conditions.

The term “target polynucleotide” refers to a polynucleotide of interest (e.g., a single gene or polynucleotide) or a group of polynucleotides (e.g., a family of polynucleotides, as described above). The target polynucleotide can designate mRNA, RNA, cRNA, cDNA or DNA. The probe is used to obtain information about the target polynucleotide: whether the target polynucleotide has affinity for a given probe. Target polynucleotides may be naturally occurring or man-made nucleic acid molecules. Also, they can be employed in their unaltered state or as aggregates with other species. Target polynucleotides may be associated covalently or non-covalently, to a binding member, either directly or via a specific binding substance. A target polynucleotide can hybridise to a probe whose sequence is at least partially complementary to a sub-sequence of the target polynucleotide.

The term “target sequence” is used herein to refer to a chosen nucleotide sequence of at most 300, 250, 200, 150, 100, 75, 50, 30, 25 or at most 15 nucleotides in length. Target sequences include sequences of at least 8, 10, 15, 25, 30, 35, 45, 50, 60, 70, 80, 90, 100, 120, 135, 150, 175, 200, 250 and 300 nucleotides in length. Non-limiting examples of target sequences include, but are not restricted to, repeat sequences such as Alu repeat sequences, conserved or non-conserved regions of gene families, introns, promoter sequences including the Hogness Box and the TATA box, signal sequences, enhancers, protein-binding domains such as a homeobox, tymobox, polymorphisms and conserved protein domains or portions thereof.

2. Combinatorial Probes

The genomes (i.e., the complete gene sequences) of organisms range in length from a few hundred nucleotides for viroids and viruses to a few billion for multicellular organisms. Conventional oligonucleotide probes, however, typically target sequences that are only 8-30 nucleotides long for detection purposes. Thus, in order to identify suitable oligonucleotide probes for use in detection of target polynucleotides, short stretches (sub-strings or sub-sequences) of the target polynucleotide sequences are considered. This may be done by converting the sequences of the target polynucleotides or of reference sequences corresponding to the target polynucleotides into all possible sub-sequences or sub-sequences of those lengths or it may be done by defining the sub-sequence that is to be considered using a “window” placed over the target polynucleotide or reference sequences. This second technique may be used to consider a set of short aligned sub-sequences from a larger alignment. Depending on the range of length of sub-sequences that are considered, some of the possible sub-sequences will overlap or contain others (FIG. 1). Conserved, substantially similar or substantially identical sequences can be found using these techniques as implemented in well know algorithms. Longer conserved regions may also be identified if substantially identical or similar sub-sequences are found to overlap or to be adjacent or in close proximity.

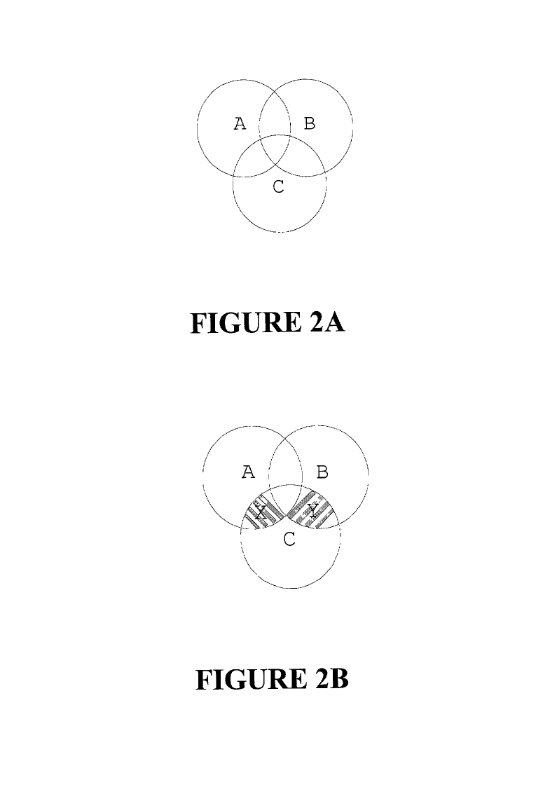

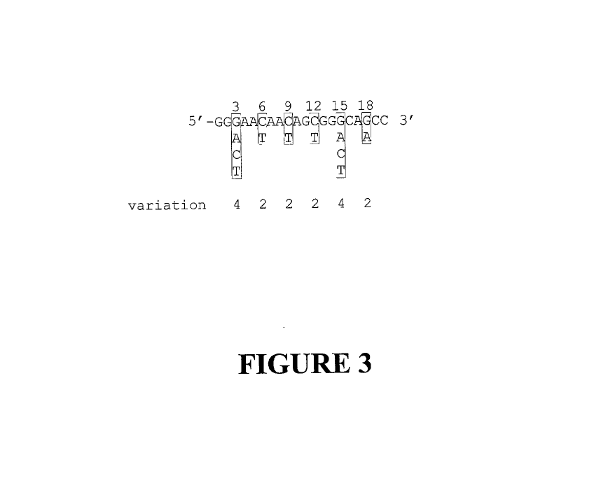

Some sub-sequences will be unique to a target polynucleotide (i.e., not found in other target polynucleotides) but many of the shorter sub-sequences from one target polynucleotide will also be found in other target polynucleotide (shared sub-sequences). Moreover, different sets of these shorter sub-sequences will be shared between different combinations of target polynucleotides (FIG. 2A) (i.e., one target polynucleotide may share some sub-sequences with another target polynucleotide but another set of sub-sequences will be shared with a third target polynucleotide and so on). It follows that probes designed from the shared sub-sequences will hybridise to more than one target polynucleotide and when probes are designed from several different shared sub-sequences the pattern of hybridisation will be complex. Such shared and unique sub-sequences form the basis of target sequences as described hereinafter.

The present invention is predicated in part on a novel strategy for decreasing the number and/or size of oligonucleotide probes required for detecting and distinguishing between a plurality of target polynucleotides. The strategy involves detecting different target polynucleotides using a set of oligonucleotide probes, which includes a collection of promiscuous probes, wherein each promiscuous probe is capable of hybridising to a predetermined sub-sequence or target sequence shared between at least two target polynucleotides.

The target polynucleotides to be detected comprise two or more target sequences, at least one of which is shared with one or more other target polynucleotides. Despite the promiscuity of a respective promiscuous probe hybridising to more than one target polynucleotide, a particular target polynucleotide can be specifically detected by detecting hybridisation thereto of at least two promiscuous probes, wherein different target polynucleotides are identified by different combinations of such probes.

For example, the instant combinatorial detection can be carried out minimally using three gene targets, e.g., targets A, B and C. These genes could be identified using three specific probes, but they could also be identified by only two probes, if these probes were designed using the sequences of two shared target sequences, x and y. A probe designed from target sequence x reacts with A, one designed from target sequence y reacts with B and both probes react with C (FIG. 2B). Furthermore, the shorter an oligonucleotide is, the greater the number of gene sequences with which it is likely to hybridise, therefore probes used in a combinatorial way can be shorter than those that are specific. Hence, efficiently designed combinatorial arrays will be comprised of fewer and typically shorter probes, than those using target-specific probes. Thus, a particular advantage of such arrays is that they will be less costly to produce. The potential savings will depend in part on the size of the set of target sequences: the larger the target sequence set the greater the potential savings will be as the number of target sequences that are available for combinatorial detection or identification is larger.

The set of probes may optionally contain non-promiscuous probes each of which is capable of hybridising to a single or unique target sequence in the plurality of target polynucleotides. In this embodiment, non-promiscuous probes and combinations of promiscuous probes are used to distinguish between the plurality of different target polynucleotides. Accordingly, a respective target polynucleotide can be specifically detected by detecting hybridisation thereto of at least two promiscuous probes, or a single non-promiscuous probe.

The above combinatorial approach is particularly useful for designing efficient sets of probes to detect, for example, all likely members of a group of related but variable genes. Large sets of probes are required if every possible sequence is to be identified specifically. However, if a combinatorial approach is used as described herein the required specificity can be obtained by using a combination of small sets of less specific (i.e., cross hybridising) or promiscuous probes.

From the foregoing, a set of probes can be designed so that a target polynucleotide would hybridise to at least two probes from the set. In one embodiment, different combinations of cross-reactive or ‘promiscuous’ probes only are used to discriminate between, and identify specifically, a plurality of target polynucleotides. In another embodiment, probes that hybridise to target sequences uniquely in concert with promiscuous probes are used to provide such discrimination and identification. The saving in the number of probes will depend on the variability of the target sequences. If a large set of specific probes is used to detect redundant sequence variation, then the number of degenerate probes that would be required is the product of the number of variations at all the variable sites in a sub-sequence. By contrast, when shorter less specific probes are used these are less variable and their number is equal only to the sum of the number of probes used for each variable site. An example of this sort is described below.

The sequences of the shared reference sequences may have been conserved during the evolution of the target polynucleotides (i.e., the target polynucleotides have some common ancestry) or they may be shared because coincidental sequence similarities have arisen through a process of convergence. Both types of shared sequences are useful for designing promiscuous probes according to the invention. Another set of target sequences that could be used would be those that are similar to varying degrees. Different target polynucleotides should contain many such similar target sequences and because under certain conditions probes will hybridise with sequences that are almost identical but not absolutely identical, some similar target sequences could be used. Useful reference sequences for guiding selection of target sequences include, but are not restricted to, those defining repeat sequences, conserved or non-conserved regions of gene families, introns or exons, promoters, signal sequences, enhancers, boxes, protein-binding domains, polymorphisms and conserved protein domains or other multinucleotide groupings of interest (e.g.,—homeoboxes, tymoboxes, etc). In one embodiment, the probe set includes probes that define the degenerate set of oligonucleotides. In addition, or as an alternative to degenerate probe sets, useful probes can contain inosine, other generic bases, or mixtures of A, C, T G especially at the third position of a codon site. In an alternate embodiment, a reference sequence defines a polymorphism. In this instance, probes interrogate the presence of individual polymorphic variants.

The combinatorial method for designing reduced sets of probes could be applied to any test or device that uses two or more probes, and it will allow significant economies or cost savings in tests or devices that use larger numbers of probes and have a broad range of target polynucleotides. The method could be used in one embodiment to improve the design of DNA micro-arrays that are used for gene expression studies, pathogen strain typing, genotype typing, diagnosis, forensics or any other use requiring that species or genes be detected, distinguished or identified. The method could also be used to improve the design of tests or devices that are based on nucleotide hybridisation but that do not use the probes in arrays or bonded to a solid matrix, that use RNA oligonucleotides or that use nucleic acid analogues for the same purpose.

Preferably, the set of probes is immobilised on one or more solid supports. An oligonucleotide probe may be immobilised to the solid support using any suitable technique. For example, Holstrom et al. (1993, Anal. Biochem. 209: 278-283) exploit the affinity of biotin for avidin and streptavidin, and immobilise biotinylated nucleic acid molecules to avidin/streptavidin coated supports. Another method which may be employed involves precoating of polystyrene or glass solid phases with poly-L-Lys or poly-L-Lys, Phe, followed by covalent attachment of either amino- or sulfhydryl-modified oligonucleotides using bifunctional cross linking reagents (Running et al., 1990, Biotechniques 8: 276-277; Newton et al., 1993, Nucleic Acids Res. 21: 1155-1162). Kawai et al. (1993, Anal. Biochem. 209: 63-69) describe an alternative method in which short oligonucleotide probes are ligated to form multimers before cloning thereof into a phagemid vector. The oligonucleotides are then immobilized onto a polystyrene plate and fixed by UV irradiation at 254 nm. Reference also may be made to a method for the direct covalent attachment of short, 5′-phosphorylated oligonucleotide primers to chemically modified polystyrene plates (Covalink™ plate, Nunc) (Rasmussen et al., 1991, Anal. Biochem. 198: 138-142). Regard may also be had to an article by O'Connell-Maloney et al. (1996, TIBTECH 14: 401-407) which discloses immobilisation of biotinylated oligonucleotides and sulfhydrylated oligonucleotides respectively to a streptavidin-coated silicon wafer and an iodoacetamide-coated silicon wafer. Also, amino-modified oligonucleotides have been immobilized on isothiocyanate-coated glass (Guo et al., 1994, Nucleic Acids Res. 22: 5456-5465) and silane-epoxide-coated wafer (Eggers et al., 1994, BioTechniques 17: 516-5240). The aforementioned methods refer to post-synthetic attachment of oligonucleotide primers to a substrate. Alternatively, the oligonucleotide primers may be synthesised in situ utilising, for example, the method of Maskos and Southern (1992, Nucleic Acids Res. 20 1679-1684) or that of Fodor et al. (supra). Suitably, the set of probes is in the form of a nucleic acid array, preferably a high-density nucleic acid array, which may optionally comprise a mixture of different but individually addressable microbeads.

It will of course be appreciated that the oligonucleotide probes used in the invention may be immobilized either directly or indirectly. For example, a probe may be adsorbed to a surface or alternatively covalently bound to a spacer molecule, which has been covalently bound to the solid support. The spacer molecule may include a latex microparticle, a protein such as bovine serum albumin (BSA) or a polymer such as dextran or poly-(ethylene glycol). Such a spacer molecule is considered to improve accessibility of the oligonucleotide primer to hybridisation of the target nucleotide sequence. Alternatively, the spacer molecule may comprise a homo-polynucleotide tail such as, for example, oligo-dT. In a preferred embodiment, the spacer molecule is 10 to 25 molecules in length.

Probes may be designed to optimise specific hybridisation to their reference sequences. For example, Drmanac et al. (U.S. Pat. No. 5,972,619) describe probes containing a core 8-mer and one of three possible variations at outer positions with two variations at each end. Such probes are represented as 5′-(A, T, G, C)(A, T, G, C) N8 (A, T, G, C)-3′. With this type of probe one does not need to discriminate the non-informative end bases (two on 5′ end, and one on 3′ end) since only the internal 8-mer is read as the probe sequence.

3. Identifying Target Sequences

The invention also contemplates a process for identifying target sequences for the preparation of a set of oligonucleotide probes as broadly defined above. In one embodiment, the process comprises searching a nucleic acid sequence database comprising the sequences of a plurality of target polynucleotides for identical target sequences that are shared between two or more of the target polynucleotides to thereby obtain a subset of shared target sequences (shared subset). Preferably, the process further comprises recording the positions in each polynucleotide sequence of all overlapping sub-sequences, for example between 8 and 30 nucleotides in length, within that sequence. In an alternate embodiment, the process further comprises recording the positions in each polynucleotide sequence of all unique sub-sequences within that sequence (unique subset). In yet another embodiment, the process further comprises sorting the target sequences from said subset(s) to obtain target sequences with substantially similar affinities for their complementary oligonucleotide probes.

Potential target sequences that are preferably identified in the sub-sequence database include, but are not restricted to:

1. Pivot sequences that preferably divide two or more target polynucleotides into two sets, one set comprising from 40-60% of the target group in which the pivot sequence is present, and the other, the remaining 60-40% of the polynucleotides, in which the pivot sequence is not present. This sorting would be done using a computational embodiment in the style of Danzig's simplex algorithm of linear programming.

2. Conserved or redundant sequences that distinguish the target group of polynucleotides from all outside the target group by being present in the target polynucleotide sequences and rare or absent in others.

Accordingly, in another embodiment, the process further comprises recording the positions in each polynucleotide sequence of any target sequences that divide two or more target polynucleotides into sets, thus defining a pivot sequence subset. In yet another embodiment, process further comprises recording the positions in each polynucleotide sequence of any target sequences that are substantially identical or conserved between related target polynucleotides. Redundant sequences corresponding to potential sequence variants of such target sequences can then be deduced to obtain a subset of redundant target sequences (redundant subset), which correspond to potentially unknown or uncharacterised target polynucleotides.

A combination of target sequences is then selected from one or more of the shared subset, the redundant subset and the pivot subset or a single target sequence is selected from the unique subset, for specifically detecting each target polynucleotide or group of target polynucleotides. In the case of detecting a putative unknown or uncharacterised member of a polynucleotide family, a predefined assemblage of target sequences is identified wherein at least one member of the combination is a redundant target sequence. The unknown or uncharacterised member would, therefore, be expected to hybridise with a predefined assemblage of oligonucleotide probes, wherein at least one probe is substantially complementary to a redundant target sequence.

In a preferred embodiment, a minimal or near minimal number of oligonucleotide probes is determined which, in different combinations, discriminate between the different target polynucleotides.

It is preferred that at least 2, more preferably at least 10, more preferably at least 50, more preferably at least 100 and still more preferably at least 1000 different combinations of target sequences are determined for specifically detecting a corresponding number of target polynucleotides.

From the foregoing, it will be appreciated that sets of probes based on pivot sequences, that divide the target polynucleotides in substantially all possible combinations, and that are of minimal or near minimal length, can be used to provide efficient probes for identifying target polynucleotides using micro-arrays. Sets of probes based on conserved sequences can be used to provide taxonomic information since they represent regions of gene families that have been inherited from a shared ancestor. Probe sequences, like those described hereinafter for potyviruses can then be deduced from such taxonomic analysis, to provide a basis for the construction of a probe array that can identify as-yet-unknown relatives of a chosen target group or family of polynucleotides. It is also envisaged that some target sequences will occur in both pivot and conserved groups, and that most of these shared sequences will be recognised as contiguous regions of shared sequences.

In practice, it is envisaged that the most efficient micro-arrays will comprise mixtures of probes identified by both pivot and conserved searching techniques, pruned after tests for sequence redundancy, and expanded to include permutations of contiguous and conserved regions so as to capture likely sequence variants of gene families.

It is also envisaged that efficient micro-arrays will not only identify known target sequences but also related sequences. Further that previously unknown polynucleotides will be recognised and initially characterised by such micro-arrays, and that the probe sequences with which unknown polynucleotides are found to hybridise can be used as primers in polymerase chain reactions to further characterise and identify such unknown polynucleotides.

4. Computer Related Embodiments

The design or construction of a set of combinatorial probes of the present invention is suitably facilitated with the assistance of a computer programmed with software, which inter alia searches a nucleic acid sequence database comprising the sequences of a plurality of target polynucleotides for identical target sequences that are shared between two or more of the target polynucleotides to thereby obtain a subset of shared target sequences (shared subset). The software determines subsequently for each target polynucleotide a combination of target sequences from said subset whose sequence information can be used to construct probes that can facilitate specific detection of that target polynucleotide. Thus, in another aspect, the invention encompasses a computer for designing the sequence of a set of combinatorial probes of the invention, wherein the computer comprises: (a) a machine readable data storage medium comprising a data storage material encoded with machine readable data, wherein the machine readable data comprises a plurality of target polynucleotides (e.g., a gene database); (b) a working memory for storing instructions for processing the machine-readable data; (c) a central-processing unit coupled to the working memory and to the machine-readable data storage medium, for processing the machine-readable data to provide identical target sequences that are shared between two or more of the target polynucleotides; and (d) an output hardware coupled to the central processing unit, for receiving said identical target sequences.

In a preferred embodiment, the computer processes said machine-readable data to provide for each target polynucleotide a combination of target sequences, which when hybridised by complementary or substantially complementary oligonucleotide probes, facilitate specific detection of that target polynucleotide. The computer may also process the machine-readable data to record positions in each polynucleotide sequence of all overlapping sub-sequences, for example between 8 and 30 nucleotides in length, within that sequence. Alternatively, or additionally, the computer may process the machine-readable data to record the positions in each polynucleotide sequence of all unique sub-sequences within that sequence (unique subset).

In a preferred embodiment, the computer processes the machine-readable data to sort the target sequences in said subset(s) to obtain target sequences with substantially similar affinities for their complementary oligonucleotide probes. Alternatively or additionally, the computer may process the machine-readable data to record the positions in each polynucleotide sequence of any target sequences that divide two or more target polynucleotides into sets, thus defining a pivot sequence subset. In an alternate embodiment, the computer may process the machine-readable data to record the positions in each polynucleotide sequence of any target sequences that are substantially identical or conserved between related target polynucleotides. The computer also may process the machine-readable data to deduce redundant sequences corresponding to potential sequence variants of such target sequences to obtain a subset of redundant target sequences (redundant subset), which correspond to potentially unknown or uncharacterised target polynucleotides.

The invention also contemplates a computer program product for designing combinatorial probes of the present invention, comprising code that receives as input sequences of target polynucleotides from one or more nucleic acid sequence databases and/or information that identifies sequences corresponding to said target polynucleotides; code that identifies potential target sequences within the target polynucleotides; code that identifies the target sequences that are shared between different target polynucleotides; optional code that identifies the target sequences that are unique to specific target polynucleotides, code that assesses every possible combination or a number of combinations of the target sequences to identify those combinations of target sequences which, when hybridised by complementary oligonucleotide probes, facilitate discrimination between different target polynucleotides; and a computer readable medium that stores the codes.

In a preferred embodiment, the computer program product further comprises code that creates a database which registers the presence or absence of possible target sequences found within respective target polynucleotides. Additionally, or alternatively, the computer program product further comprises code that identifies substantially identical or conserved sequences between the target sequences and code that identifies redundant sequence variants of said substantially identical target sequences, wherein said redundant sequence variants are registered as target sequences.

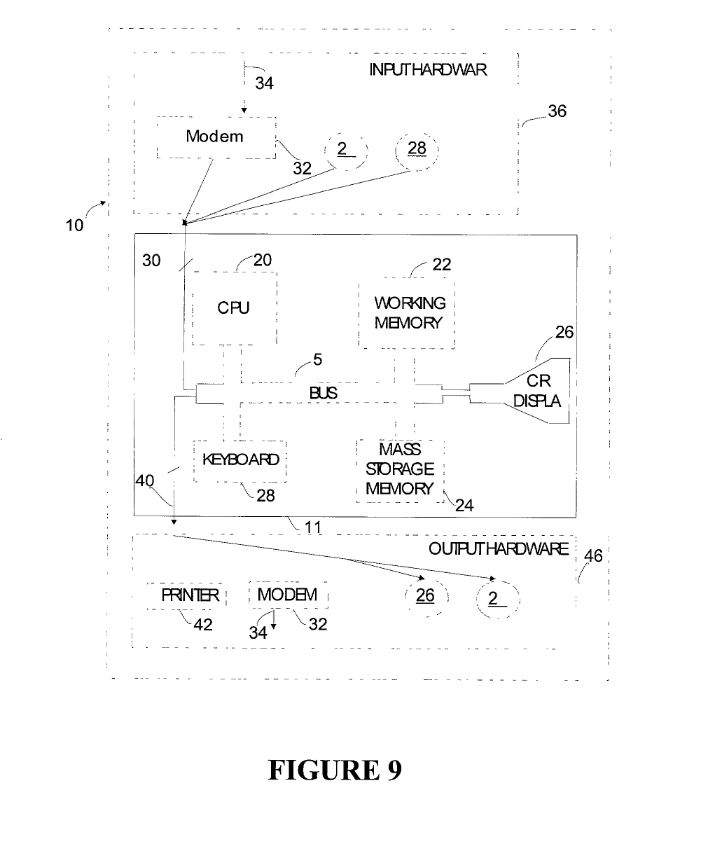

A version of these embodiments is presented in FIG. 9, which shows a system 10 including a computer 11 comprising a central processing unit (“CPU”) 20, a working memory 22 which may be, e.g., RAM (random-access memory) or “core”memory, mass storage memory 24 (such as one or more disk drives or CD-ROM drives), one or more cathode-ray tube (“CRT”) display terminals 26, one or more keyboards 28, one or more inputs lines 30, and one or more output lines 40, all of which are interconnected by a coventional bidirectional system bus 50.

Input hardware 36, coupled to computer 11 by input lines 30, may be implemented in a variety of ways. For example, machine-readable data may be inputted via the use of a modem or modems 32 connected by a telephone line or dedicated data line 34. Alternatively or additionally, the input hardwear 36 may comprise CD. Alternatively, ROM drives or disk drives 24 in conjunction with display 26, keyboard 28 may also be used as an input device.

Output hardwear 46, coupled to computer 11 by outputs lines 40, may similarly be implemented by conventional devices. By way of example, output hardwear 46, may include CRT display terminals 26 for displaying a synthetic polynucleotide sequence or a synthetic polypeptide sequence a describe herein. Output hardwear might also include a printer 42, so that hard copy may be produce, or a disk drive 24, to store system output for later use.

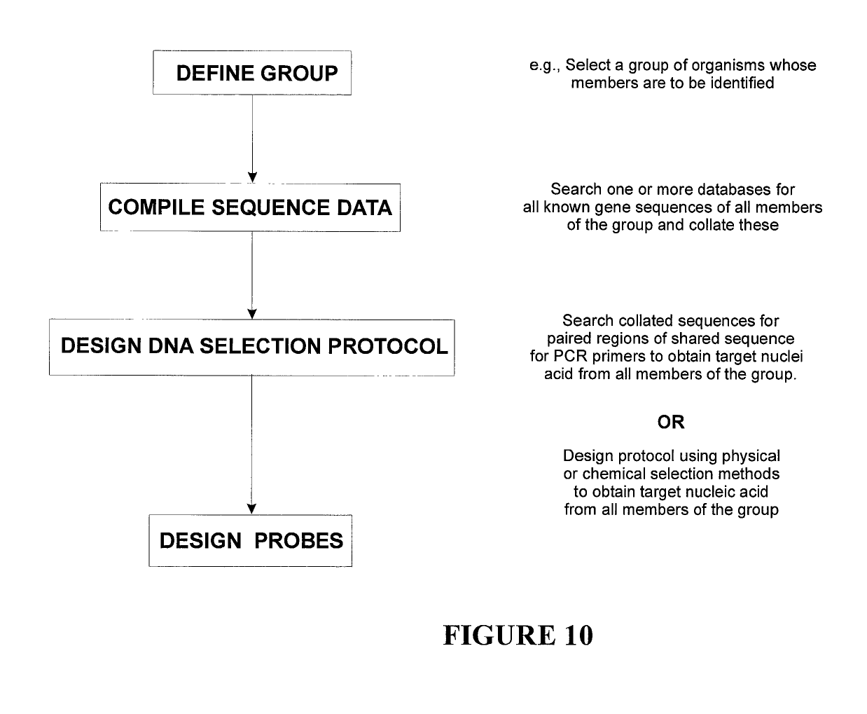

In operation CPU 20 coordinates the use of the various input and outputs devices 36, 46 coordinates data accesses from mass storage 24 and accesses to and from working memory 22, and determines the sequence of data processing steps. A number of programs may be used to process the machine readable data of this invention. Exemplary programs may use for example the steps outlined in the flow diagram illustrated in FIG 10. Broadly, these steps include (1) selecting a group of entities to be identified (e.g., a group of organisms, a family of related polynucleotides etc): (2) compiling sequence data for those entities; (3) identifying target sequences that are shared between those entities to provide a subset of shared sequences; (4) deriving potential oligonucleotides sequences (oligos), which can be used as probes for detecting and distinguishing members of the group; (5) preparing primary “taxon x oligo ” matrix; (6) deducing a meta “taxon pair-oligo” matrix (7) identifying a “minimum set cover” of oligos using “greedy strategy”; (8) identifying replicate sets of identical probes from oligos of step (7); and (9) evaluating discriminatory power of the probes.

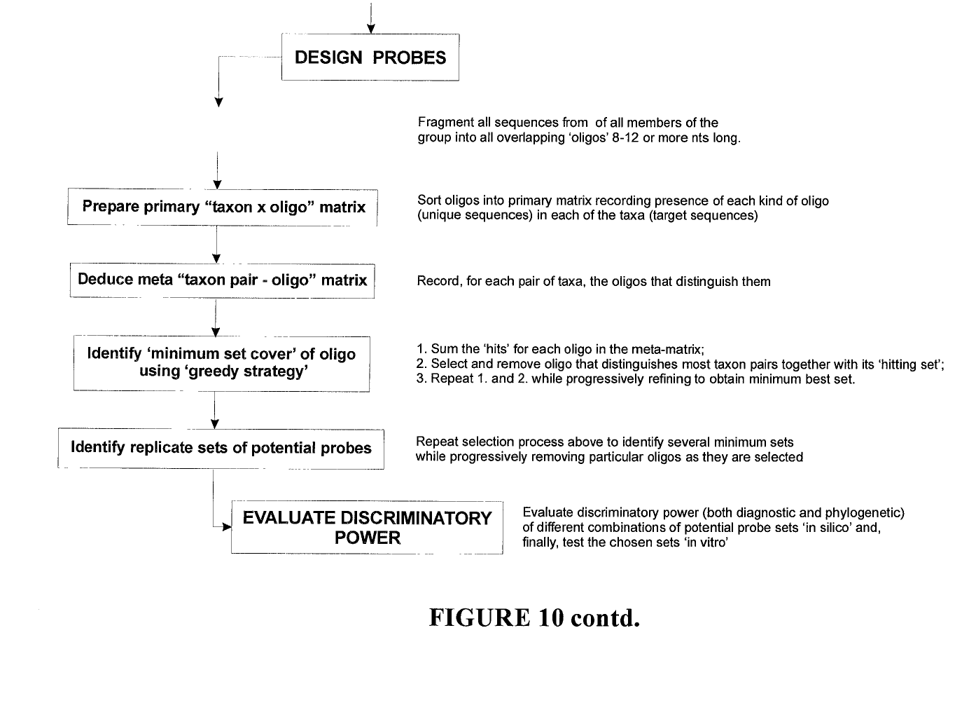

FIG. 11 shows a cross section of a magnetic data storage medium 100 which can be encoded with machine readable data, or set of instruction, for a set of probes of the invention, which can be carried out by a system such as system 10 of FIG 9. Medium 100 can be a coventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24. The magnetic domains of coating 102 of medium 100 are polarised or oriented so as to encode in manner which may be conventional, machine readable data such as that described herein, for execution by a system such as system 10 of FIG. 9.

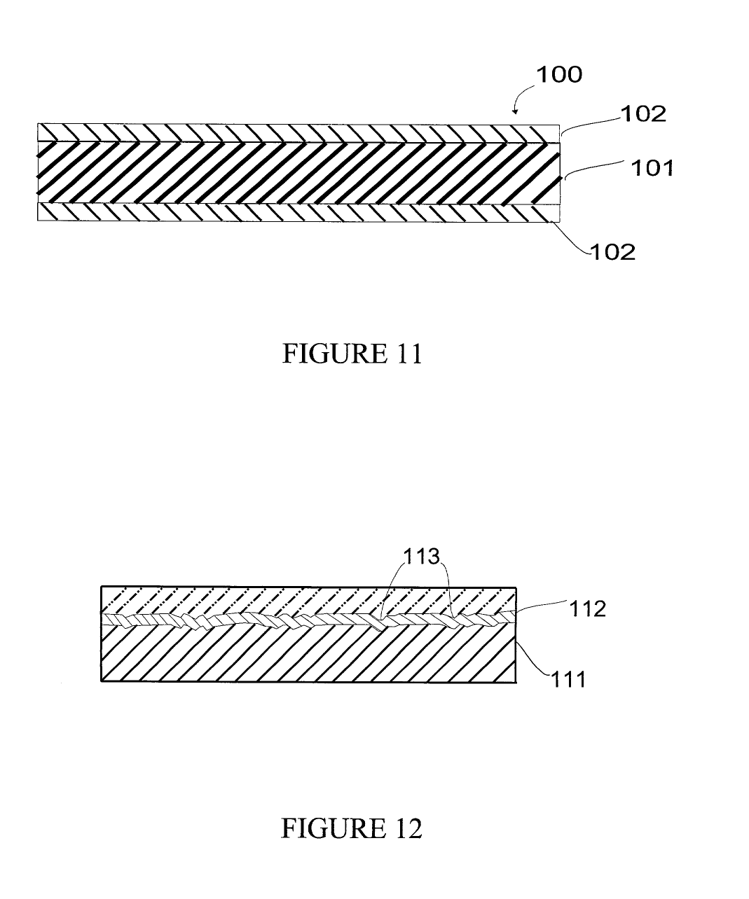

FIG. 12 shows a cross section of an optically readable data storage medium 110 which also can be encoded with such a machine-readable data, or set of instructions, for designing a synthetic molecule of the invention, which can be carried out by a system such as system 10 of FIG. 9. Medium 110 can be a conventional compact disk read only memory (CD-ROM) or a rewritable medium such as a magneto-optical disk, which is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarisation of laser light reflected from coating 112. The arrangement of the domains encodes the data as described above.

5. Screening Method

The invention also provides a method for detecting a plurality of different target polynucleotides using a set of probes as broadly described above. The method comprises exposing the probes to a test sample suspected of containing one or more of said target polynucleotides under conditions favouring specific hybridisation. Suitable test samples that may be used in the method may include extracts of double or single stranded nucleic acids obtained from archaeal, eubacterial or eukaryotic origin. For example, such extracts may be obtained from cells, tissues or materials derived from plants, fungi, bacteria or animals as well as materials derived from viruses, satellite viruses, viroids and similar non-cellular organisms.

Sample extracts of DNA or RNA, either single or double-stranded, may be prepared from fluid suspensions of biological materials, or by grinding biological materials, or following a cell lysis step which includes, but is not limited to, lysis effected by treatment with SDS (or other detergents), osmotic shock, guanidinium isothiocyanate and lysozyme. Suitable DNA, which may be used in the method of the invention, includes genomic DNA or cDNA. Such DNA may be prepared by any one of a number of commonly used protocols as for example described in CURRENT PROTOCOLS IN MOLECULAR BIOLOGY (Ausubel, et al., eds.) (John Wiley & Sons, Inc. 1995), and MOLECULAR CLONING. A LABORATORY MANUAL (Sambrook, et al., eds.) (Cold Spring Harbor Press 1989). Sample extracts of RNA may be prepared by any suitable protocol as for example described in CURRENT PROTOCOLS IN MOLECULAR BIOLOGY (supra), MOLECULAR CLONING. A LABORATORY MANUAL (supra) and Chomczynski and Sacchi (1987, Anal. Biochem. 162 156, hereby incorporated by reference).

Suitable RNA, which may be used in the method of the invention, includes messenger RNA, complementary RNA transcribed from DNA (cRNA) or genomic or subgenomic RNA. Such RNA may be prepared using standard protocols as for example described in the relevant sections of Ausubel, et al. (supra) and Sambrook, et al. (supra).

The genomic DNA or cDNA may be fragmented, for example, by sonication or by treatment with restriction endonucleases. Suitably, the genomic DNA or cDNA is fragmented such that resultant DNA fragments are of a length greater than the length of the immobilized oligonucleotide probe(s) but small enough to allow rapid access thereto under suitable hybridisation conditions. Alternatively, fragments of genomic DNA or cDNA may be selected and amplified using a suitable nucleotide amplification technique, involving appropriate random or specific primers. Such amplification techniques are well known to those of skill in the art and include, for example, PCR (Saiki et al, 1988, supra), Strand Displacement Amplification (SDA) (U.S. Pat. No. 5,422,252, Little et al.), Rolling Circle Replication (RCR) (Liu et al., 1996, J. Am. Chem. Soc. 118: 1587-1594; International Application Publication No WO 92/01813), Nucleic Acid Sequence Based Amplification (NASBA) (Sooknanan et al., 1994, Biotechniques 17 1077-1080) and Q-β replicase amplification (Tyagi et al., 1996, Proc. Natl. Acad. Sci. USA 93: 5395-5400).

Usually the target polynucleotides or fragments thereof are detectably labelled so that their hybridisation to individual probes can be determined. In this regard, the target polynucleotides or fragments may have one or more reporter molecules associated therewith. The reporter molecule may be selected from a group including a chromogen, a catalyst, an enzyme, a fluorochrome, a chemiluminescent molecule, a bioluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like. Especially preferred labels of this type include large colloids, for example, metal colloids such as those from gold, selenium, silver, tin and titanium oxide. In one embodiment in which an enzyme is used as a direct visual label, biotinylated bases are incorporated into a target polynucleotide. Hybridisation is detected by incubation with streptavidin-reporter molecules.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. Nos. 5,573,909 (Singer et al), 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218. Commercially available fluorescent labels include, for example, fluorescein phosphoramidites such as Fluoreprime (Pharmacia), Fluoredite (Millipore) and FAM (Applied Biosystems International).

Radioactive reporter molecules include, for example, ³²p, which can be detected by a X-ray or phosphoimager techniques.

The hybrid-forming step can be performed under suitable conditions for hybridising oligonucleotide probes to test nucleic acid including DNA or RNA. In this regard, reference may be made, for example, to NUCLEIC ACID HYBRIDIZATION, A PRACTICAL APPROACH (Homes and Higgins, eds.) (IRL press, Washington D.C., 1985). In general, whether hybridisation takes place is influenced by the length of the oligonucleotide probe and the polynucleotide sequence under test, the pH, the temperature, the concentration of mono- and divalent cations, the proportion of G and C nucleotides in the hybrid-forming region, the viscosity of the medium and the possible presence of denaturants. Such variables also influence the time required for hybridisation. The preferred conditions will therefore depend upon the particular application. Such empirical conditions, however, can be routinely determined without undue experimentation.

Preferably high discrimination hybridisation conditions are used. For example, reference may be made to Wallace et al. (1979, Nucl. Acids Res. 6: 3543) who describe conditions that differentiate the hybridisation of 11 to 17 base long oligonucleotide probes that match perfectly and are completely homologous to a target sequence as compared to similar oligonucleotide probes that contain a single internal base pair mismatch. Reference also may be made to Wood et al. (1985, Proc. Natl. Acid. Sci. USA 82: 1585) who describe conditions for hybridisation of 11 to 20 base long oligonucleotides using 3 M tetramethyl ammonium chloride wherein the melting point of the hybrid depends only on the length of the oligonucleotide probe, regardless of its GC content. In addition, Drnanac et al. (supra) describe hybridisation conditions that allow stringent hybridisation of 6-10 nucleotide long oligomers, and similar conditions may be obtained most readily by using nucleotide analogues such as ‘locked nucleic acids (Christensen et al., 2001 Biochem J 354: 481-4).

Generally, a hybridisation reaction can be performed in the presence of a hybridisation buffer that optionally includes a hybridisation optimising agent, such as an isostabilising agent, a denaturing agent and/or a renaturation accelerant. Examples of isostabilising agents include, but are not restricted to, betaines and lower tetraalkyl ammonium salts. Denaturing agents are compositions that lower the melting temperature of double stranded nucleic acid molecules by interfering with hydrogen bonding between bases in a double stranded nucleic acid or the hydration of nucleic acid molecules. Denaturing agents include, but are not restricted to, formamide, formaldehyde, dimethylsulphoxide, tetraethyl acetate, urea, guanidium isothiocyanate, glycerol and chaotropic salts. Hybridisation accelerants include heterogeneous nuclear ribonucleoprotein (hnRP) Al and cationic detergents such as cetyltrimethylammonium bromide (CTAB) and dodecyl trimethylammonium bromide (DTAB), polylysine, spermine, spermidine, single stranded binding protein (SSB), phage T4 gene 32 protein and a mixture of ammonium acetate and ethanol. Hybridisation buffers may include target polynucleotides at a concentration between about 0.005 nM and about 50 nM, preferably between about 0.5 nM and 5 nM, more preferably between about 1 nM and 2 nM

A hybridisation mixture containing the target polynucleotides is placed in contact with the array of probes and incubated at a temperature and for a time appropriate to permit hybridisation between the target sequences in the target polynucleotides and any complementary probes. Contact can take place in any suitable container, for example, a dish or a cell designed to hold the solid support on which the probes are bound. Generally, incubation will be at temperatures normally used for hybridisation of nucleic acids, for example, between about 20° C. and about 75° C., example, about 25° C., about 30° C., about 35° C., about 40° C., about 45° C., about 50° C., about 55° C., about 60° C., or about 65° C. For probes longer than 14 nucleotides, 20° C. to 50° C. is preferred. For shorter probes, lower temperatures are preferred. A sample of target polynucleotides is incubated with the probes for a time sufficient to allow the desired level of hybridisation between the target sequences in the target polynucleotides and d any complementary probes. For example, the hybridisation may be carried out at about 45° C.+/−10° C. in formamide for 1-2 days.

After the hybrid-forming step the probes are washed to remove any unbound nucleic acid with a hybridisation buffer, which can typically comprise a hybridisation optimising agent in the same range of concentrations as for the hybridisation step. This washing step leaves only bound target polynucleotides. The probes are then examined to identify which probes have hybridised to a target polynucleotide.

The hybridisation reactions are then detected to determine which of the probes has hybridised to a corresponding target sequence. Depending on the nature of a reporter molecule associated with a target polynucleotide, a signal may be instrumentally detected by irradiating a fluorescent label with light and detecting fluorescence in a fluorimeter; by providing for an enzyme system to produce a dye which could be detected using a spectrophotometer; or detection of a dye particle or a coloured colloidal metallic or non metallic particle using a reflectometer; in the case of using a radioactive label or chemiluminescent molecule employing a radiation counter or autoradiography. Accordingly, a detection means may be adapted to detect or scan light associated with the label which light may include fluorescent, luminescent, focussed beam or laser light. In such a case, a charge couple device (CCD) or a photocell can be used to scan for emission of light from a probe:target polynucleotide hybrid from each location in the micro-array and record the data directly in a digital computer. In some cases, electronic detection of the signal may not be necessary. For example, with enzymatically generated colour spots associated with nucleic acid array format, as herein described, visual examination of the array will allow interpretation of the pattern on the array. In the case of a nucleic acid array, the detection means is preferably interfaced with pattern recognition software to convert the pattern of signals from the array into a plain language genetic profile. In a preferred embodiment, the set of probes is in the form of a nucleic acid array and detection of a signal generated from a reporter molecule on the array is performed using a ‘chip reader’. A detection system that can be used by a ‘chip reader’ is described for example by Pirrung et al (U.S. Pat. No. 5,143,854). The chip reader will typically also incorporate some signal processing to determine whether the signal at a particular array position or feature is a true positive or maybe a spurious signal. Exemplary chip readers are described for example by Fodor et al (U.S. Pat. No., 5,925,525). Alternatively, when the array is made using a mixture of individually addressable kinds of labelled microbeads, the reaction may be detected using flow cytometry.

6. Data Analysis

The hybridisation data are then processed to determine which probes have formed hybrids. In a preferred embodiment, a digital computer is employed to correlate specific positional labelling on the array with the presence of any of the target sequences for which the probes have specificity of interaction. The positional information is directly converted to a database indicating what sequence interactions have occurred. Data generated in hybridisation assays is most easily analysed with the use of a programmable digital computer. The computer program product generally contains a readable medium that stores the codes. Certain files are devoted to memory that includes the location of each feature and all the target sequences known to contain the sequence of the oligonucleotide probe at that feature. Computer methods for analysing hybridisation data from nucleic acid arrays is taught in PCT publication No WO97/29212 and EP publication 95307476.2. In a preferred embodiment the programmable computer would contain specialist software code and register data derived from the entire sequence database, or containing that part of the entire sub-sequence database that is relevant to the particular probe array, and from the pattern of hybridisation will assess the probability that particular target sequences were present in the tested DNA sample.

The computer program product can also contain code that receives as input hybridisation data from a hybridisation reaction between a target sequence and an oligonucleotide probe. The computer program product can also include code that processes the hybridisation data. Data analysis can include the steps of determining, for example, the fluorescence intensity as a function of substrate position from the data collected, removing “outliers” (data deviating from a predetermined statistical distribution), and calculating the relative binding affinity of the target sequences from the remaining data. The resulting data can be displayed as an image with colour in each region varying according to the light emission or binding affinity between target sequences and probes therein.

In one embodiment, the amount of binding at each address is determined by examining the on-off rates of the hybridisation. For example, the amount of binding at each address is determined at several time points after the nucleic acid sample is contacted with the array. The amount of total hybridisation can be determined as a function of the kinetics of binding based on the amount of binding at each time point. Persons of skill in the art can easily determine the dependence of the hybridisation rate on temperature, sample agitation, washing conditions (e.g., pH, solvent characteristics, temperature) in order to maximise conditions for hybridisation rate and signal to noise.

The computer program product also can include code that receives instructions from a programmer as input. The computer program product may also transform the data into a format for presentation.

In one embodiment, the computer program product for processing hybridisation data comprises code that identifies for each target polynucleotide a combination of features in an oligonucleotide array whose probes facilitate specific detection of that polynucleotide; code that receives as input hybridisation data from hybridisation reactions between sample polynucleotides and the oligonucleotide probes in the array; code that processes the hybridisation data to determine whether the sample polynucleotides comprise any of the target polynucleotides by searching for hybridisation patterns that match any of the predefined combinations of target sequences; and a computer readable medium that stores the codes. It is not necessary to identify the sequence of respective oligonucleotide probes in each feature of the array. In this respect, the hybridisation analysis software only requires as input which combination of features in the array corresponds to a particular target polynucleotide. However, in a preferred embodiment, the computer program product comprises code that receives as input the sequence of an oligonucleotide probe in each feature of an oligonucleotide array and code that receives as input a database that contains information on the presence or absence of target sequences in target polynucleotides.

Preferably the computer program product further comprises code that deduces the probability that the detected pattern of hybridisation indicates the presence of a target polynucleotide.

The database of target sequences would be regularly up-dated and the part of it relevant to each particular set of probes forming each micro-array would also be updated for those using particular commercial applications of the invention.

In order that the invention may be readily understood and put into practical effect, particular preferred embodiments will now be described with reference to the following examples.

### EXAMPLES

### Example 1

Combinatorial probes for detection of different strains of potato virus Y

Illustrated in this example is the use of probe combinations to detect all members of a variable gene family using, as an example, the gene sequences of the potyviruses, the largest genus of the family Potyviridae. The Potyviridae is the largest and one of the best-studied plant virus families, species of which cause significant losses in many crops throughout the world. At least 400 potyviruses are known, and they comprise about one quarter of all known plant viruses.

Several different strategies could be used to design the probes for DNA micro-arrays that could detect and distinguish between different potyviruses. The most direct, but most inefficient, strategy would be to convert the genomic RNAs of all known potyviruses into cloned DNAs and to use a sample of each of those DNAs as the probes in a DNA micro-array. Many tests would have to be done to check the specificity or otherwise of those probes for individual potyviruses, and there is no guarantee that any novel potyviruses, discovered subsequently, would be detected by a DNA micro-array constructed from those components.

A much better strategy would be to use the genomic sequences of potyviruses in the international gene sequence databases to design specific probes based on shared sequences. At present around 75 potyvirus genomes have been fully sequenced (c. 10,000 nucleotides each) and recorded in the databases together with partial sequence of many others. Sequence analysis has shown that the sequences of these genomes are similar to a greater or lesser extent. Thus, a set of probes designed for the shared regions should detect the presence of all known potyviruses, and would also be likely to detect all as-yet-undescribed potyviruses. An array of cloned potyvirus cDNAs described above would probably not have this last property.

The most conserved part of all potyvirus genomic sequences is the so-called ‘B motif’ of their polymerase gene and is a stretch 20 nucleotides long (FIG. 3). This shared region contains fourteen nucleotide ‘regions’ that do not vary and six that do (FIG. 3); at four regions one or other of two nucleotides are found in different species, and at two regions one or other of all four nucleotides are found. To date many of the different combinations of the nucleotides recorded at the variable regions in the sequence have been found in different potyviruses, but not all. However, in designing a micro-array to detect both known and unknown potyviruses, it will be prudent to include all combinations of the variable nucleotides, and this is illustrated in the following example.

When the set of related sequences described in FIG. 3 is checked against the current international sequence databases (1.7×10⁹ nucleotides; May 2000), every one of the sequenced potyvirus genomes is matched by one of the variant sequences, and only one sequence in this set matches a non-potyvirus sequence, which is a human gene sequence of unknown function. To construct a micro-array of probes that would encompass all this variation, so that each potyvirus could be specifically detected by a single probe, one would need 256 probe sequences (4×2×2×2×4×2=256 combinations) as illustrated in FIG. 4.

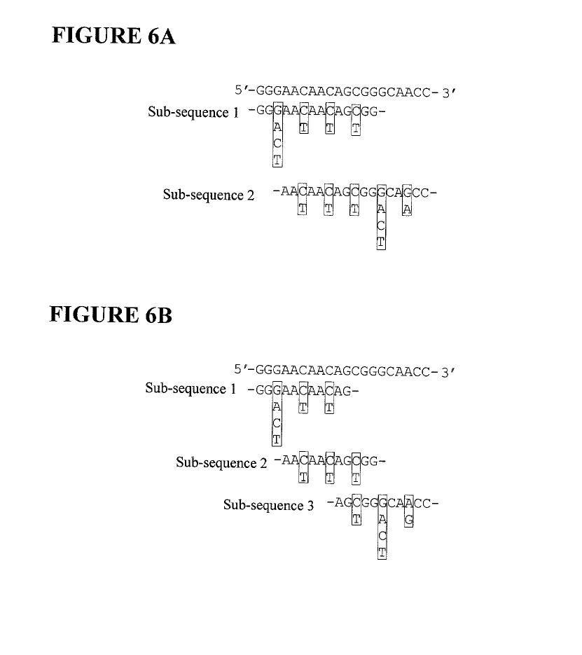

Using a micro-array of this design the variants of the genome region encoding the ‘potyvirus B-motif’ in the six strains of potato virus Y (PVY) would hybridise with the probes illustrated in the three diagrams in FIG. 5. Interestingly the probe that would hybridise with PVY-CO (FIG. 5C) would also hybridise with bean yellow mosaic potyvirus strain S, but not strain MB.

The same potyvirus genomes would, however, be detected more efficiently using micro-arrays designed by the combinatorial approach mentioned above and such arrays would be more informative as they will be more discriminating. The presence of the conserved B-motif region of potyviruses described above could be detected by fewer shorter probes if two overlapping sub-groups of sequences derived from the 20-nucleotide long sequence were used (FIG. 6A). One sub-group would be only 14 nucleotides long and would omit the last six nucleotides of the full motif, and, therefore, the sub-group would be of 32 sequences (4×2×2×2=32 combinations). The other sub-group would omit the first 3 nucleotides of the full motif, would, therefore, be 17 nucleotides long and would thus be of 64 sequences (2×2×2×4×2=64 combinations). A micro-array of these two sub-groups would therefore consist of 96 probes, namely about one third of the number of probes required by the full 20 nucleotide motif. When this array is used in a test, the presence of a potyvirus polymerase B-motif region will be indicated by hybridisation to at least one probe from each sub-group. cDNAs derived from some potyviruses would bind to the same probes in one sub-group but different probes in the other sub-group and hence, an array designed from these sequences would work in a combinatorial way.

Even greater savings would accrue if the B-motif were represented by three overlapping stretches, each 11 nucleotides long (FIG. 6B). All possible combinations of the conserved B-motif sequence could then be represented by just 40 probes, and thus, the number of probes required would decrease to 16% (40/256), and the number of nucleotides required in the probes would decrease to 9% of the 256 probe array (440/5120). When an array carrying the three sets of shorter sequences is used in a test, the presence of a potyvirus B-motif region will be indicated by hybridisation to at least one probe from each of the three sub-groups.

Arrays designed using the two or three sub-groups of B motif sequences would be less specific than an array consisting of probes with the complete 20-nucleotide long sequences. However, their specificity could be augmented, perhaps to an even greater level than the larger array, by including additional probes based on other regions of the potyvirus genome.

Two other conserved regions in all potyvirus genomes that could be used are shown in FIGS. 6C and D. The first of these, which encodes the ‘WCIEN-motif’ of the virion protein, could be subdivided, like the B-motif gene, into two overlapping regions; one omitting the last three nucleotides and the other the first five. The resulting two sub-groups, 13 and 11 nucleotides long, would require 48 probes to represent all combinations of the variable sequence positions. The second, which encodes the ‘NEVD-motif’ of the cylindrical inclusion protein, would also require a single set of 48 probes to represent all known variants. If a micro-array was designed using these three additional conserved sequences together with the two B motif sub-group sequences shown in FIG. 6B then the five subsets would together comprise 136 rather than 256 probes (53%) and 1492 nucleotides rather than 5120 (29%).

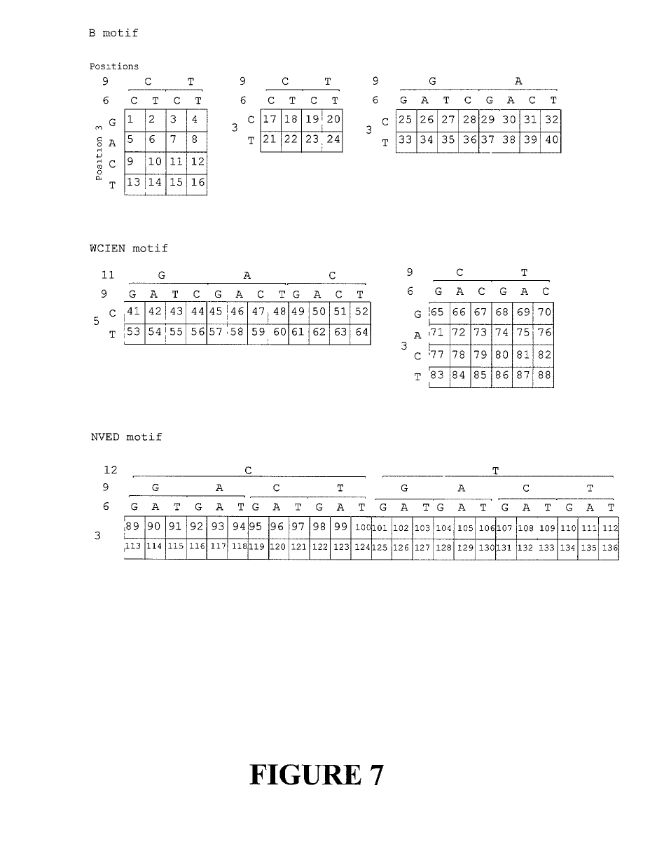

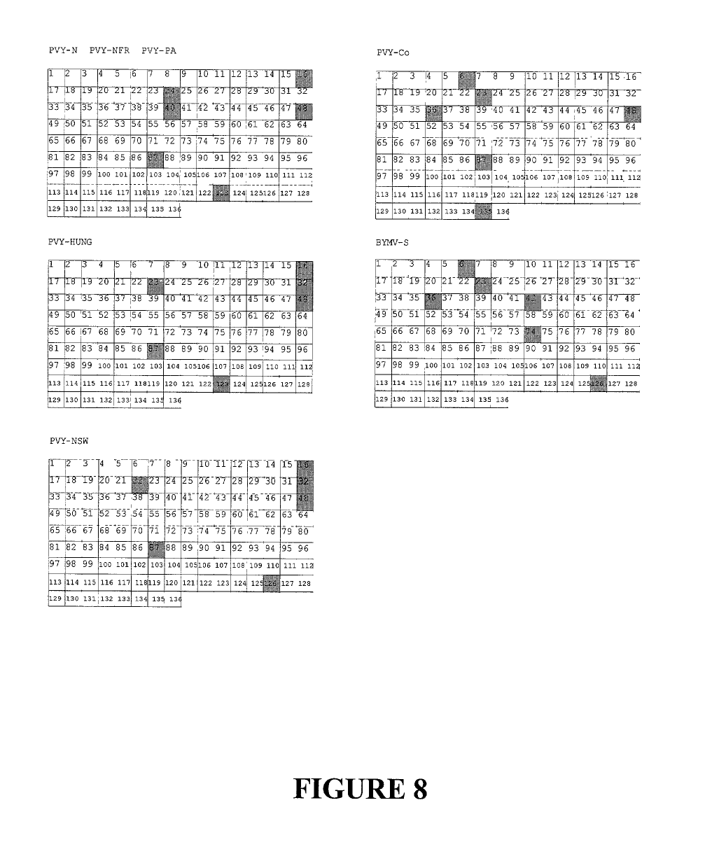

A micro-array comprising these five sub-groups of sequences is described in FIG. 7. For comparison, the hybridisation pattern in FIG. 8 is shown between such an array and the cDNAs of the virus genes used in the example of the array with the complete 20 nucleotide long B-motif probe sequences (FIG. 5). The combinatorial array would be similarly capable of detecting any potyvirus cDNA but could also be used to distinguish between the PVY-Hung and NSW strains and between PVY-Co and BYMV. The larger array would not have those capabilities.

It is difficult to estimate the specificity of combinatorial probe sets because of the complexity and biases of gene sequences, and because their specificity would depend in practice on the source of the cDNA, and hence the likely contaminants. However, it could be estimated computationally using the international gene sequence databases, or parts of them, and it might be found that adequate specificity could be provided by just three or four sub-groups rather than five. The potyvirus example given above would, minimally, halve the number of probes required for a diagnostic micro-array and decrease the cost even more, and the saving could, of course, be greater still if the micro-array had other gene targets that shared the probes in other combinations.

The example explained above using known genomic sequences of the potyviruses involves the use of overlapping sections of three regions of their genomes, however the combinatorial strategy can be applied, with equal value to non-contiguous (non-overlapping) sequences. These could be found conveniently using appropriate computer algorithms.

### Example 2

Process of Identifying Combinatorial Probes

Illustrative in this example is one embodiment of the process of the invention for identifying sequences useful for producing combinatorial probes for detecting a plurality of organisms.

Sequences to be used as combinatorial probes can be identified using known sequences (e.g., published in a nucleic acid sequence database) relating to target polynucleotides (e.g., a gene or group of genes or transcripts relating thereto) of a plurality of organisms of interest. Finding the “minimum set” of sub-sequences to cover likely variation in the target polynucleotides and to be used as a probe set is a “Nondeterministic Polynomial time (NP)-complete” problem, and algorithms for the identification of suitable target sequences can be based on principles discussed for example in: Garey, M. R. and Johnson, D. S. (1979). Computers and intractability: A guide to the theory of NP-completeness. W. H. Freeman & Co, San Fransisco; Crescenzi, P. and Kann, V. (eds). A compendium of NP optimization problems; and Halldorsson, M. (sub-ed); Graph Theory: Covering and partitioning. http://www.nada.kth.se/˜viggo/problemlist/compendium.html

A preferred process for the identification of suitable target sequences for distinguishing a set of organisms of interest, which is summarised in FIG. 10, can proceed by the following computational stages:

1). A nucleic acid sequence database is searched for sequences of a selected genomic region present in the target set of organisms, which might define, for example, a plurality of “taxa”. By way of example, the selected region may comprise sequences ZZ which are delimited by, and can be amplified in PCR using a pair of redundant PCR primers (i.e., mixtures of primers that hybridise with all known species of the set), for example all the recorded polymerase genes of influenza (orthomyxo) viruses. These sequences are complied for stage (2).

2). The compiled sequences are fragmented into sets of shorter overlapping nucleotide sequences or oligonucleotide sequences (oligos) that are, ideally, 8-12 nucleotides long, but may be 6 or more nucleotides long.

3). All oligos of a particular size are sorted into a primary “taxon x oligo” matrix; initially different matrices are constructed for each oligo size class. In each matrix is recorded the presence or absence of each kind of oligo in each of the taxa.

4). A “meta-taxon pair x oligo” matrix (or meta-matrix.) is then constructed from each primary matrix by comparing all taxon pairs in the primary matrix and recording, for each pair, whether or not they are distinguished by each oligo.

5). The “minimum set” of oligos to distinguish the target sequences is then derived from the meta-matrix, using the standard “greedy strategy”:

a). The oligo that distinguishes most taxa in the meta-matrix is identified by summing the number of hits for each oligo in the meta-matrix;

b). That oligo is then removed from the meta-matrix together with its “hitting set”, namely all the pairs of taxa that it distinguishes;

c). This process is repeated until hitting sets that include all or most taxa have been found; usually 12 or more in number;

d). As, typically, more than one “best” oligo is identified at each summation step, the algorithm iteratively and progressively tests all possible sets to identify the best minimum set by swapping oligos at each iteration. Other criteria can also be used to select the oligos that are likely (for physico-chemical reasons) to make the best probes, for example, those that are of similar composition and those that are not nested subsequences of one another.

Each working set of probes can use several minimum sets of oligos discovered in this way. At least 5 sets are usually required to ensure the accuracy of identification, especially as a single individual minimum set may not uniquely identify all taxa in the set. A working set may also include oligos of more than one length class.

The disclosure of every patent, patent application, and publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A set of oligonucleotide probes for detecting a plurality of different target polynucleotides, wherein a respective target polynucleotide corresponds to a single polynucleotide or a group of related polynucleotides, said set including a collection of different promiscuous probes, wherein a respective promiscuous probe is capable of hybridising to a target sequence shared between at least two of said target polynucleotides, wherein at least one target polynucleotide comprises at least two target sequences shared between other target polynucleotides, and wherein a predefined combination of promiscuous probes is capable of hybridising to said at least two target sequences, said predefined combination providing specificity of detection of said at least one target polynucleotide.

2. The set of probes of claim 1, comprising a plurality of different predefined combinations of probes, each providing specificity of detection of a different target polynucleotide.

3. The set of probes of claim 1, further comprising at least one non-promiscuous probe that is capable of hybridising to a unique target sequence of a single target polynucleotide.

4. The set of probes of claim 1, comprising at least one probe that is capable of hybridising to a pivot sequence, which divides two or more polynucleotides into distinct groups.

5. The set of probes of claim 1, comprising at least one degenerate oligonucleotide probe which is capable of hybridising to a redundant target sequence.

6. The set of probes of claim 1, wherein the probes are immobilised on a solid support.

7. The set of probes of claim 6, wherein the probes are in the form of a nucleic acid array.

8. The set of probes of claim 7, wherein the probes are in the form of a high-density nucleic acid array.

9. The set of probes of claim 6, wherein the probes are linked to the support via a spacer.

10. A method for detecting a plurality of different target polynucleotides using the set of probes of claim 1, said method comprising:
exposing said probes to a test sample suspected of containing one or more of said target polynucleotides under stringent hybridisation conditions;
detecting which probes have hybridised to polynucleotides in said test sample; and
processing the hybridisation data to determine which of said predefined combinations of probes has hybridised to said polynucleotides to thereby determine whether the test sample comprises any of said target polynucleotides.

11. The method of claim 10, wherein said stringent conditions favour high discrimination hybridisation.

12. The method of claim 10, further comprising analysing whether any of said target polynucleotides in said test sample corresponds to a phenotype-determining target polynucleotide.

13. The method of claim 12, further comprising diagnosing a phenotype of a patient from which said test sample was derived based on the phenotype-determining target polynucleotide(s) present in the test sample.

14. The method of claim 10, wherein said processing is performed by a programmable digital computer.

15. A method for detecting an unknown or uncharacterised member of a polynucleotide family using the set of probes of claim 1, said method comprising:
exposing said probes to a test sample under stringent hybridisation conditions;
detecting which probes have hybridised to polynucleotides in said test sample; and
processing the hybridisation data to determine which combinations of probes have hybridised to polynucleotides in said test sample, and whether any of said combinations is different to at least one predefined combination of probes that hybridise to known target sequences, wherein the presence of a different combination of oligonucleotide probes is indicative of the presence of said unknown or uncharacterised member.

16. The method of claim 15, wherein the different combination of oligonucleotide probes corresponds to a hypothetical predefined combination of probes belonging to a predefined assemblage.

17. The method of claim 16, wherein the hypothetical predefined combination of probes comprises at least one degenerate oligonucleotide probe that is capable of hybridising to a redundant target sequence.

18. A process of identifying a set of target sequences from a plurality of known target polynucleotides for designing a set of oligonucleotide probes for detecting said target polynucleotides, wherein a respective target polynucleotide corresponds to a single polynucleotide or a group of related polynucleotides, said set including a collection of different promiscuous probes, wherein a respective promiscuous probe is capable of hybridising to a target sequence shared between at least two of said target polynucleotides, wherein at least one target polynucleotide comprises at least two target sequences shared between other target polynucleotides, and wherein a predefined combination of promiscuous probes is capable of hybridising to said at least two target sequences, said predefined combination providing specificity of detection of said at least one target polynucleotide, said process comprising:
searching a nucleic acid sequence database comprising the sequences of said target polynucleotides for identical target sequences that are shared between two or more of said target polynucleotides to thereby obtain a subset of shared target sequences; and
determining for each target polynucleotide a combination of target sequences from said subset which, when hybridised by complementary or substantially complementary oligonucleotide probes, facilitate specific detection of that target polynucleotide.

19. The process of claim 18, further comprising:
determining a minimal or near minimal number of promiscuous oligonucleotide probes, which in different combinations, discriminate between the different target polynucleotides.

20. The process of claim 18, further comprising:
sorting the target sequences from said subset to obtain a subset of pivot sequences which divide two or more polynucleotides into distinct groups.

21. The process of claim 18, further comprising:
searching the database for sequences that are unique to respective target polynucleotides to thereby obtain a subset of unique target sequences;
determining for each target polynucleotide a target sequence from said unique subset, or a combination of target sequences from said unique subset and said shared subset which, when hybridised by complementary or substantially complementary oligonucleotide probe(s), facilitate(s) specific detection of that target polynucleotide.

22. The process of claim 21, further comprising:
determining a minimal or near minimal number of promiscuous probes which, in different combinations, together with one or more non-promiscuous probes, discriminate between the different target polynucleotides.

23. The process of claim 18, further comprising:
searching the database for target sequences that are substantially identical or conserved between related target polynucleotides; and
deducing redundant sequences corresponding to potential sequence variants of said target sequences to thereby obtain a subset of redundant target sequences which correspond to potentially unknown or uncharacterised target polynucleotides; and
determining for each target polynucleotide a target sequence from said redundant subset, or a combination of target sequences from said shared subset and/or said redundant subset which, when hybridised by complementary or substantially complementary oligonucleotide probe(s), facilitate(s) specific detection of that target polynucleotide

24. The process of any one of claims 18, 20, 21 and 23, further comprising:
sorting target sequences from said subset(s) to obtain target sequences with substantially similar affinities for their complementary or substantially complementary oligonucleotide probes.

25. A process of identifying a set of target sequences from a plurality of known target polynucleotides for designing a set of oligonucleotide probes for detecting said target polynucleotides, wherein a respective target polynucleotide corresponds to a single polynucleotide or a group of related polynucleotides, said set including a collection of different promiscuous probes, wherein a respective promiscuous probe is capable of hybridising to a target sequence shared between at least two of said target polynucleotides, wherein at least one target polynucleotide comprises at least two target sequences shared between other target polynucleotides, and wherein a predefined combination of promiscuous probes is capable of hybridising to said at least two target sequences, said predefined combination providing specificity of detection of said at least one target polynucleotide, said process comprising:
searching a nucleic acid sequence database comprising the sequences of said target polynucleotides for identical target sequences that are shared between two or more of said target polynucleotides to thereby obtain a subset of shared target sequences;
optionally searching the database for sequences that are unique to respective target polynucleotides to thereby obtain a subset of unique target sequences;
searching the database for target sequences that are substantially identical or conserved between related target polynucleotides and deducing redundant sequences corresponding to potential sequence variants of said target sequences to thereby obtain a subset of redundant target sequences which correspond to potentially unknown or uncharacterised target polynucleotides.
determining for each target polynucleotide a target sequence from said unique subset or from said redundant subset, or a combination of target sequences from said shared subset and/or from said redundant subset which, when hybridised by complementary or substantially complementary oligonucleotide probe(s), facilitate specific detection of that target polynucleotide.

26. The process of claim 25, further comprising:
sorting the target sequences from said redundant subset, from said shared subset and, if any, from said unique subset to obtain target sequences with substantially similar affinities for their complementary or substantially complementary oligonucleotide probes.

27. The process of claim 25, further comprising:
determining a minimal or near minimal number of promiscuous probes which, in different combinations, together with one or more non-promiscuous probes, discriminate between the different target polynucleotides.

28. The process of claim 18 or claim 25, wherein said process is performed by a digital computer.

29. A computer program product for identifying a set of target sequences for designing a set of oligonucleotide probes according to claim 1, comprising code that receives as input sequences of target polynucleotides in one or more nucleic acid sequence databases and/or information that identifies sequences corresponding to said target polynucleotides; code that identifies potential target sequences within the target polynucleotides; code that creates a database that registers the presence or absence of possible target sequences found within respective target polynucleotides; code that identifies the target sequences that are shared between different target polynucleotides; optional code that identifies the target sequences that are unique to specific target polynucleotides, code that assesses every possible combination or a number of combinations of the target sequences to identify those combinations of target sequences which, when hybridised to complementary oligonucleotide probes, will facilitate discrimination between different target polynucleotides; and a computer readable medium that stores the codes.

30. The computer program product of claim 29, further comprising code that identifies substantially identical or conserved sequences between the target sequences and code that identifies redundant sequence variants of said substantially identical target sequences, wherein said redundant sequence variants are registered as target sequences.

31. A computer program product for processing hybridisation data using the set of oligonucleotide probes according to claim 1, comprising code that identifies for each target polynucleotide a combination of features in an oligonucleotide array whose probes facilitate specific detection of that polynucleotide; code that receives as input hybridisation data from hybridisation reactions between sample polynucleotides and the oligonucleotide probes in the array; code that processes the hybridisation data to determine whether the sample polynucleotides comprise any of the target polynucleotides by searching for hybridisation patterns that match any of the predefined combinations or predefined assemblages of target sequences; and a computer readable medium that stores the codes.

32. The computer program product of claim 31, further comprising code that receives as input the sequence of an oligonucleotide probe in each feature of an oligonucleotide array and code that receives as input a database that contains information on the presence or absence of target sequences in target polynucleotides.

33. The computer program product of claim 31, further comprising code that deduces the probability that the detected pattern of hybridisation indicates the presence of a target polynucleotide.


SEQUENCE LISTING (26 sequences)

SEQ ID NO: 1 - DNA, 10 bp, Artificial Sequence
  Synthetic polynucleotide
agctcattga

SEQ ID NO: 2 - DNA, 9 bp, Artificial Sequence
  Synthetic polynucleotide
agctcattg

SEQ ID NO: 3 - DNA, 9 bp, Artificial Sequence
  Synthetic polynucleotide
gctcattga

SEQ ID NO: 4 - DNA, 8 bp, Artificial Sequence
  Synthetic polynucleotide
agctcatt

SEQ ID NO: 5 - DNA, 8 bp, Artificial Sequence
  Synthetic polynucleotide
gctcattg

SEQ ID NO: 6 - DNA, 8 bp, Artificial Sequence
  Synthetic polynucleotide
ctcattga

SEQ ID NO: 7 - DNA, 20 bp, Artificial Sequence
  Synthetic polynucleotide
ggnaayaayagyggncarcc

SEQ ID NO: 8 - DNA, 15 bp, Artificial Sequence
  Synthetic polynucleotide
ggaaaacagggcacc

SEQ ID NO: 9 - DNA, 15 bp, Artificial Sequence
  Synthetic polynucleotide
ggaaaatagggcacc

SEQ ID NO: 10 - DNA, 15 bp, Artificial Sequence
  Synthetic polynucleotide
gggaaaaagggcacc

SEQ ID NO: 11 - DNA, 15 bp, Artificial Sequence
  Synthetic polynucleotide
ggaaaaaagggcacc

SEQ ID NO: 12 - DNA, 15 bp, Artificial Sequence
  Synthetic polynucleotide
ggcaaaaagggcacc

SEQ ID NO: 13 - DNA, 15 bp, Artificial Sequence
  Synthetic polynucleotide
ggtaaaaagggcacc

SEQ ID NO: 14 - DNA, 15 bp, Artificial Sequence
  Synthetic polynucleotide
ggaacaaagggcacc

SEQ ID NO: 15 - DNA, 15 bp, Artificial Sequence
  Synthetic polynucleotide
ggaataaagggcacc

SEQ ID NO: 16 - DNA, 20 bp, Artificial Sequence
  Synthetic polynucleotide
gggaacaacagcgggcaacc

SEQ ID NO: 17 - DNA, 14 bp, Artificial Sequence
  Synthetic polynucleotide
ggnaayaayagygg

SEQ ID NO: 18 - DNA, 17 bp, Artificial Sequence
  Synthetic polynucleotide
aayaayagyggncarcc

SEQ ID NO: 19 - DNA, 20 bp, Artificial Sequence
  Synthetic polynucleotide
gggaacaacagcgggcaacc

SEQ ID NO: 20 - DNA, 11 bp, Artificial Sequence
  Synthetic polynucleotide
ggnaayaayag

SEQ ID NO: 21 - DNA, 11 bp, Artificial Sequence
  Synthetic polynucleotide
aayaayagygg

SEQ ID NO: 22 - DNA, 11 bp, Artificial Sequence
  Synthetic polynucleotide
agyggncarcc

SEQ ID NO: 23 - DNA, 16 bp, Artificial Sequence
  Synthetic polynucleotide
ggtgyatngavaaygg

SEQ ID NO: 24 - DNA, 13 bp, Artificial Sequence
  Synthetic polynucleotide
ggtgyatngavaa

SEQ ID NO: 25 - DNA, 11 bp, Artificial Sequence
  Synthetic polynucleotide
atngavaaygg

SEQ ID NO: 26 - DNA, 12 bp, Artificial Sequence
  Synthetic polynucleotide
aaygadgtngay